SEQUENCE LISTING (10 sequences)

SEQ ID NO: 1 - protein, 154 aa, synthetic construct
MGRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 2 - protein, 153 aa, Homo sapiens
APVRSLNCRIWDVNQKTFYLRNNQLVAGYLQGPNVNLEEKFSMSFVQGEESNDKIPVALGLKEKNLYLSCVLKDDKPTLQLESVDPKNYPKKKMEKRFVFNKIEINNKLEFESAQFPNWFLCTAMEADQPVSLTNMPDEGVMVTKFYMQFVSS

SEQ ID NO: 3 - protein, 145 aa, Homo sapiens
APVRSLNCRIWDVNQKTFYLRNNQLVAGYLQGPNVNLEEKFSMSFVQGEESNDKIPVALGLKEKNLYLSCVLKDDKPTLQLESVDPKNYPKKKMEKRFVFNKIEINNKLEFESAQFPNWFLCTAMEADQPVSLTNMPDEGVMVTK

SEQ ID NO: 4 - protein, 880 aa, synthetic construct
SERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVEKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 118 aa, synthetic construct [sequence omitted]

SEQ ID NO: 6 - protein, 110 aa, Homo sapiens
EIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPFTFGPGTKVDIKRTV

SEQ ID NO: 7 - protein, 120 aa, synthetic construct [sequence omitted]

SEQ ID NO: 8 - protein, 110 aa, synthetic construct
DIQMTQSTSSLSASVGDRVTITCRASQDISNYLSWYQQKPGKAVKLLIYYTSKLHSGVPSRFSGSGSGTDYTLTISSLQQEDFATYFCLQGKMLPWTFGQGTKLEIKRTV

SEQ ID NO: 9 - protein, 113 aa, synthetic construct
VQLVQSGAEVKKPGSSVKVSCKASGYAFTSSWMNWVRQAPGQGLEWMGRIYPGDGNTHYAQKFQGRVTLTADKSTSTAYMELSSLRSEDTAVYYCGEGYLDPMDYWGQGTLVT

SEQ ID NO: 10 - protein, 107 aa, synthetic construct
DIQMTQSPSSLSASVGDRVTITCQASQGINNYLNWYQQKPGKAPKLLIHYTSGLHAGVPSRFSGSGSGTDYTLTISSLEPEDVATYYCQQYSILPWTFGGGTKVEIK

(19) United States
(12) Patent Application Publication
(10) Pub. No.: US 2026/0232817 A1
(43) Pub. Date: Aug. 13, 2026

(54) Activatable Drug Conjugates and Therapeutic Applications Thereof

(71) Applicants: California Institute of Technology, Pasadena, CA (US); The Regents of the University of California, Oakland, CA (US)

(72) Inventors: Daniel M. Schwartz, Pasadena, CA (US); Dennis A. Dougherty, Pasadena, CA (US); Christopher B. Marotta, Pasadena, CA (US); April G. Huang, Pasadena, CA (US); Adam Oskowitz, Oakland, CA (US); Melroy X. Miranda, Oakland, CA (US)

(21) Appl. No.: 19/155,877

(22) PCT Filed: Feb. 16, 2024
(86) PCT No.: PCT/US2024/016154

§ 371 Date: Aug. 12, 2025

Related U.S. Application Data
(60) Provisional application No. 63/446,776, filed on Feb. 17, 2023.
(60) Provisional application No. 63/525,922, filed on Jul. 10, 2023.

(51) Int. Cl. A61K 47/64; A61K 41/00; A61N 5/06; A61P 9/14

(52) U.S. Cl. CPC A61K 47/64; A61K 41/0057; A61N 5/0601; A61N 5/062; A61P 9/14

(57) ABSTRACT

Provided and described herein are photoactivatable drug conjugates useful for treating extracellular matrix degradation. The photoactivatable drug conjugates generally use targetable delivery of a photosensitizer (e.g., capable of generating singlet oxygen) to sites of extracellular matrix degradation and/or tissue degeneration, wherein photoactivation of the photosensitizer increases extracellular matrix crosslinking and/or tissue strength.

### CROSS-REFERENCE

This application claims the benefit of U.S. Provisional Application No. 63/446,776 filed on Feb. 17, 2023, and U.S. Provisional Application No. 63/525,922 filed on Jul. 10, 2023, each of which is incorporated by reference herein in its entirety.

### BACKGROUND

Abdominal aortic aneurysms (AAA) are a common form of vascular aneurysms with significant morbidity. Each year over 200,000 new patients are diagnosed with AAA in the US alone, and AAA rupture is a notable cause of death worldwide. If a patient with AAA ruptures, the mortality rate exceeds 50%, Arterial aneurysms are a localized, abnormal dilation of a blood vessel. These aneurysms emerge as the result of a persistent degradation of cellular structures within the arterial wall. As aneurysms progress and enlarge the structural integrity of the vessel wall continues to degrade, and as aneurysms grow and they are more likely to rupture, which can lead to life-threatening bleeding.

### SUMMARY

There is currently no method to slow or halt the growth of aneurysms. The only available therapy for aneurysms is surgical replacement or exclusion of the blood vessel (e.g., using endovascular stent-graft prosthesis). These treatments are reserved for a minority of patients whose aneurysms reach a critical size threshold when the risk of rupture exceeds the risk from an operation. Consequently, most patients with AAA have no treatment options.

Aneurysm growth can be characterized by a cycle of progressive tissue destruction, extracellular matrix breakdown and chronic inflammation that results in progressive weakening of the arterial wall. Arterial wall stability, in certain instances, relies on fibrillar collagens in media and adventitia. In larger blood vessels such as the aorta, wall elasticity relies in certain instances, on elastin and collagen fibers. The ECM of blood vessels appears to have an important role in regulating aneurysm progression. This occurs through two intertwined mechanisms. First, fibrillar collagen and elastin progressively breakdown. Second, immune cells infiltrate into the ECM and start a cycle of chronic progressive tissue destruction. Damage to the ECM is chemotactic and pro-inflammatory while immune cells release cytokines that further breakdown the ECM.

A method that prevented or slowed aneurysm growth by strengthening the ECM in blood vessels would fundamentally change the treatment of patients that have aneurysms. A treatment that breaks the cycle of ECM breakdown could potentially halt or reverse the disease.

The photoactivatable drug conjugates, systems, and methods described here provide a method for strengthening the extracellular matrix of damaged tissue therefore improving tissue integrity and making it more resistant to further degradation. The targetable photoactivatable drug conjugates, systems, and methods described here have the potential unlock new methods for treating aneurysms, vascular disease and other disease states where tissue degradation results in reduced function.

Provided and described herein are photoactivatable drug conjugates useful, in certain embodiments, for treating aneurysms. Also provided and described herein are photoactivatable drug conjugates useful, in certain embodiments, for treating extracellular matrix degradation and the progression of aneurysm growth. The photoactivatable drug conjugates generally use targetable delivery of a photosensitizer (e.g., capable of generating singlet oxygen) to sites of extracellular matrix degradation and/or tissue degeneration, wherein photoactivation of the photosensitizer increases extracellular matrix crosslinking and/or tissue strength. In addition to providing targetable and/or site-specific delivery of the photosensitizer, the compositions and methods described herein enable increased extracellular matrix crosslinking and/or tissue repair. In certain embodiments, the photoactivatable drug conjugates and systems allow for effective dose thresholds to be achieved within a target tissue, allowing for lower serum or plasma concentrations of the photosensitizer and photoactivatable drug conjugate. In certain embodiments, the photoactivatable drug conjugates and systems increase the therapeutic window for achieving effective treatment. As further provided and described herein, the photoactivatable drug conjugates are useful for the treatment of diseases, disorders, and injury characterized by tissue degeneration, for example, aortic aneurysms.

In some embodiments, provided herein are photoactivatable drug conjugates comprising anakinra attached to a photosensitizer that generates singlet oxygen when exposed to light at a wavelength of about 600 nm to about 1000 nm. In certain embodiments, the photosensitizer is attached (e.g., covalently coupled) to the targeting moiety via a linker.

In some embodiments, provided herein are photoactivatable drug conjugates comprising a photosensitizer attached to a targeting moiety, wherein the targeting moiety binds an inflamed tissue. In certain embodiments the inflamed tissue is within the site of an aneurysm. In some embodiments, the photosensitizer generates singlet oxygen when exposed to light at a wavelength within the tissue window. In some embodiments, the photosensitizer generates singlet oxygen when exposed to light at a wavelength of about 350 nanometers (nm) to about 1,000 nanometers (nm). In some embodiments, the photosensitizer generates singlet oxygen when exposed to light at a wavelength of about 650 nanometers (nm) to about 950 nanometers (nm).

In some embodiments, provided herein are photoactivatable drug conjugates comprising a photosensitizer attached to a targeting moiety, wherein the targeting moiety binds to a cell surface receptor such as a cytokine receptor. In certain embodiments, the cell is within an inflamed tissue.

In certain embodiments, the cell is a non-malignant cell. In certain embodiments, the targeting moiety is an antibody. In certain embodiments, the targeting moiety is a ligand. In certain embodiments, the photosensitizer is attached (e.g., covalently coupled) to the targeting moiety via a linker. In some embodiments, the photosensitizer generates singlet oxygen when exposed to light at a wavelength within the tissue window, the photosensitizer generates singlet oxygen when exposed to light at a wavelength of about 650 nanometers (nm) to about 950 nanometers (nm).

In certain embodiments, the targeting moiety is selected from: an antibody or antibody fragment, a protein, or a ligand. In certain embodiments, the targeting moiety is a protein. In certain embodiments, the targeting moiety is an antibody or a derivative of an antibody such as an antibody fragment or a single chain variable fragment (scFv). In certain embodiments, the targeting moiety is a ligand.

In some embodiments, the targeting moiety is selected from: anakinra, EBI-005, rilonacept, canakinumab, gevokizumab, or LY2189102. In certain embodiments, the targeting moiety is anakinra. In certain embodiments, the targeting moiety is EBI-005?. In certain embodiments, the targeting moiety is rilonacept. In certain embodiments, the targeting moiety is canakinumab. In certain embodiments, the targeting moiety is gevokizumab. In certain embodiments, the targeting moiety is LY2189102.

In some embodiments, the targeting moiety comprises an amino acid sequence having at least 85% sequence identity to any one of SEQ ID NO: 1, wherein the targeting moiety binds IL1R.

In some embodiments, the linker or a linker precursor used to form the linker comprises a functional group that allows for attachment of the linker or linker precursor to any of the targeting and photosensitizers described herein. In certain embodiments, the linker or a linker precursor used to form the linker comprises 6-maleimidocaproyl (MC), Maleimide-DOTA, maleimidopropanoyl (MP), alanine-phenylalanine (AP), p-aminobenzyloxycarbonyl (PAB), N-succinimidyl 4-(2-pyridylthio) pentanoate (SPP), N-succinimidyl 4-(N-maleimidomethyl) cyclohexane-1-carboxylate (SMCC), N-succinimidyl (4-iodo-acetyl) aminobenzoate (SIAB), valine-citrulline (VC), 6-maleimidocaproyl-valine-citrulline (MC-VC), 6-maleimidocaproyl-valine-citrulline-p-aminobenzyloxycarbonyl (MC-VC-PAB), N-succinimidyl-1-carboxylate-valine-citrulline-p-aminobenzyloxycarbonyl (SC-VC-PAB), 6-maleimidocaproyl-polyethylene glycol-valine-citrulline (MC-PEG4-VC), 6-maleimidocaproyl-polyethylene glycol-valine-alanine (MC-PEG4-VA), or MC-PEG8-VC-PAB.

In certain embodiments, the photosensitizer is methylene blue. In some embodiments, provided herein are photoactivatable drug conjugates comprising anakinra attached to a photosensitizer that generates singlet oxygen when exposed to light at a wavelength of about 600 nm to about 1000 nm.

In some embodiments, the anakinra comprises an amino acid sequence having at least 85% sequence identity to any one of SEQ ID NO: 1, wherein the anakinra binds IL1R. In some embodiments, the anakinra comprises an amino acid sequence having at least 90% sequence identity to any one of SEQ ID NO: 1, wherein the anakinra binds IL1R.

In some embodiments, provided and described herein are photodynamic therapy systems comprising: a photoactivatable drug conjugate (e.g., any one of the photoactivatable drug conjugates described herein); a light delivery unit; and a light generating unit comprising a light source. In certain embodiments, the light delivery unit comprises an optical applicator. In certain embodiments, the optical applicator comprises a catheter configured to deliver light (e.g., a laser catheter and/or light-emitting diode catheter). In certain embodiments, the catheter contains optical fibers to deliver the light. In certain embodiments, the light delivery unit is attached to the light generator and facilitates the transmission of light (e.g., a laser beam).

In some embodiments, the photosensitizer is selected from: porfimer sodium, a porphyrin, a chlorin, a pheophorbide, a bacteriopheophorbide, a phthalocyanine, an anthraquinone, a phenothiazine, a xanthene, or a cyanine. In certain embodiments, the photosensitizer is porfimer sodium. In certain embodiments, the photosensitizer is a porphyrin. In certain embodiments, the photosensitizer is a chlorin. In certain embodiments, the photosensitizer is a pheophorbide. In certain embodiments, the photosensitizer is a bacteriopheophorbide. In certain embodiments, the photosensitizer is a phthalocyanine. In certain embodiments, the photosensitizer is an anthraquinone. In certain embodiments, the photosensitizer is a phenothiazine. In certain embodiments, the photosensitizer is a xanthene. In certain embodiments, the photosensitizer is a cyanine. In certain embodiments, the photosensitizer is selected from an photosensitizer capable of being activated in the blood (e.g., within the tissue window).

In certain embodiments, the light generating unit is capable of generating light having a wavelength of about 350 nm to about 1000 nm. In certain embodiments, the light generating unit generates light having a wavelength of about 600 nm to about 1000 nm. In certain embodiments, the light delivery unit is capable of delivering light to a tissue of an individual. In certain embodiments, the light delivery unit is capable of delivering light to an organ, a joint, a blood vessel, a tendon, or a fascia. In certain embodiments, the light delivery unit is capable of delivering light to the aorta. In certain embodiment, the light delivery unit is a standard of care light delivery unit (e.g., an FDA approved catheter of delivering light to a tissue within an individual).

In some embodiments, provided are methods treating extracellular matrix (ECM) degradation in an individual, the method comprising: (a) administering to any one of the photoactivatable drug conjugates described herein; and (b) photoactivating the photosensitizer. In certain embodiments, extracellular matrix (ECM) degradation is measured by tissue degradation. In certain embodiments, tissue degradation comprises a loss of tissue (e.g., amount of tissue or density of tissue). In certain embodiments, the loss of tissue is measured by ultrasound (e.g., high resolution ultrasound), magnetic resonance elastography magnetic resonance imaging or computed tomography (CT) including CT angiography (CTA) (e.g., providing information that serves as a surrogate for tissue degradation. In certain embodiments, extracellular matrix (ECM) degradation is measured by an increased rate of tissue degradation. In certain embodiments, extracellular matrix (ECM) degradation is measured by an increased rate of tissue degradation. In certain embodiments, extracellular matrix (ECM) degradation is measured by an increased loss of tissue thickness or density. In certain embodiments, extracellular matrix (ECM) degradation is measured by changes in tensile strength.

In certain embodiments, photoactivating the photosensitizer increases crosslinking of the extracellular matrix (ECM) or extracellular matrix (ECM) degradation products. In certain embodiments, photoactivating the photosensitizer reduces and/or inhibits tissue degradation. In certain embodiments, photoactivating the photosensitizer prevents tissue degradation. In certain embodiments, photoactivating the photosensitizer reduces and/or inhibits the rate of tissue degradation. In certain embodiments, photoactivating the photosensitizer reduces and/or inhibits a loss of tissue thickness.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the invention are utilized, and the accompanying drawings of which:

FIGS. 1A, 1B, 1C, 1D, and 1E show data demonstrating that photodynamic therapy (PDT) using methylene blue (MB) as a photosensitizer inhibits the induction of AAA. Abdominal aortas of mice were treated topically with methylene blue and exposed to light with indicated controls and were harvested on week 9 after induction of AAA using active elastase. Vehicle group are mice which have undergone AAA induction surgery with active elastase and treated with vehicle only and considered as positive controls. Sham group are mice undergoing AAA surgery that are treated with heat-inactivated elastase and considered healthy controls. FIG. 1A shows quantification of the weekly percent growth in maximal diameter of infrarenal abdominal aortas measured by micro-ultrasound imaging (MUI) using the B mode in indicated groups (n=8-15 per group). Measurements done weekly for 9 weeks following induction of aneurysms and treatment. Data were analyzed by one-way ANOVA followed by the Kruskal Wallis post hoc test. ***P<0.001. FIG. 1B shows quantification of the maximal diameter of infrarenal abdominal aortas measured by MUI and normalized to body-weight (BW) in indicated groups (n=8-15 per group). Data were analyzed by one-way ANOVA followed by the Kruskal Wallis post hoc test. ***P<0.001. FIG. 1C shows representative images of abdominal aortas visualized by MUI using the B mode in indicated groups. The aneurysm is highlighted by a white circle. FIG. 1D shows representative images of the macroscopic features of AAA formation in indicated groups. FIG. 1E shows quantification of the maximal diameter of infrarenal abdominal aortas measured by micrometry normalized to BW in indicated groups (n=6-15 per group). Data were analyzed by one-way ANOVA followed by the Kruskal Wallis post hoc test. AAA indicates abdominal aortic aneurysm; BW, body weight; MUI, micro-ultrasound imaging; Veh, vehicle (HBSS); MB, methylene blue; Veh+MB+Light, photodynamic therapy (PDT).

FIGS. 2A, 2B, and 2C show data demonstrating photodynamic therapy (PDT) using methylene blue (MB) as a photoactivator modulates immune cell infiltration and apoptosis in aneurysmal tissue from mice undergoing AAA induction and concurrently treated with PDT post-induction of AAA. Abdominal aortas of mice were topically treated with methylene blue and exposed to light with indicated controls and were harvested on week 9 after induction of AAA. FIG. 2A shows representative aortic aneurysm cross-sections from the indicated groups stained with Verhoeff-Van Gieson (VVG) stain for Elastin, Masson Trichrome for type I and type III collagen, CD3 antibody stain for T-cells and CD68 antibody stain for macrophages imaged by brightfield and polarized light. For illustrative purposes, a magnification of 20× was used for vehicle group, 50× was used for MB PDT group and 100× was used for sham group to account for the variable size of whole aortic cross-sections. FIG. 2B shows a semi-quantitative grade of elastin degradation in the aortic wall from aortic aneurysms sections of mice treated with vehicle, PDT, and healthy Sham mice. FIG. 2C shows IL1B, IL-6, and IL-10 expression from serum of mice assessed by ELISAs in indicated groups (n=7-12 per group). Data were analyzed by Student t test. **P<0.01; ***P<0.001. AAA indicates abdominal aortic aneurysm; VVG staining, Verhoeff-Van Gieson staining; Trichrome, Massons Trichrome staining, Veh, vehicle (HBSS); MB, methylene blue; Veh+MB+Light, photodynamic therapy.

FIGS. 3A, 3B, 3C, 3D, and 3E show data demonstrating that MB PDT reduces the growth of AAA and expands the therapeutic window for treating an aneurysm. MB PDT was administered to mice with pre-established and progressive aneurysms 21 days after the initial aneurysm induction surgery with active elastase applied topically on the aorta. Vehicle group are mice which have undergone AAA induction surgery with active elastase applied topically on the aorta and vehicle only applied topically at the same region. This group is considered as positive controls. Sham group are mice undergoing AAA surgery with topically applied heat-inactivated elastase and considered healthy controls. FIG. 3A shows quantification of the weekly percent growth in maximal diameter of infrarenal abdominal aortas measured by micro-ultrasound imaging (MUI) using the B mode in indicated groups (n=10-15 per group). Measurements done weekly for 9 weeks following induction of aneurysms. Treatment done on Day 21 post-induction of aneurysms. Data were analyzed by one-way ANOVA followed by the Kruskal Wallis post hoc test. *P<0.001. FIG. 3B shows quantification of the maximal diameter of infrarenal abdominal aortas measured by MUI and normalized to body-weight (BW) in indicated groups (n=10-15 per group). Data were analyzed by one-way ANOVA followed by the Kruskal Wallis post hoc test. ***P<0.001. FIG. 3C shows representative images of abdominal aortas visualized by MUI using the B mode in indicated groups. The aneurysm is highlighted by a white circle. FIG. 3D shows representative images of the macroscopic features of AAA formation in indicated groups. FIG. 3E shows quantification of the maximal diameter of infrarenal abdominal aortas measured by micrometry normalized to body-weight (BW) in indicated groups (n=10-15 per group). Data were analyzed by one-way ANOVA followed by the Kruskal Wallis post hoc test. AAA indicates abdominal aortic aneurysm; BW, body weight; MUI, micro-ultrasound imaging; Veh, vehicle (HBSS); MB, methylene blue; Veh+MB+Light, photodynamic therapy.

FIGS. 4A and 4B show peptide mapping analysis of differential drug-to-antibody ratio (DAR) in anakinra methylene blue conjugates. FIG. 4A shows the sequence of Anakinra, where conjugated lysines are marked as *. FIG. 4B shows MS spectra for Ana-MB conjugate-site peptides for differential DAR levels. Ana-MB, anakinra conjugated to methylene blue; DAR, drug-to-antibody ratio.

FIGS. 5A, 5B, 5C, 5D, 5E, 5F, 5G, and 5H show data demonstrating that Targeted Delivery of Methylene Blue (anakinra attached to methylene blue, Ana-MB) to Inflammatory Aneurysmal Tissue Improves Efficacy of Photodynamic Therapy. Vehicle group are mice which have undergone AAA induction surgery with active elastase applied topically on the aorta and vehicle only applied topically at the same region. This group is considered as positive controls. Sham group are mice which have undergone AAA surgery treated heat-inactivated elastase and considered healthy controls. FIG. 5A shows representative images of IL-1R expression in cross-sections of aneurysms in vehicle and sham mice, with a higher density of IL-1R expression in vehicle mice. FIG. 5B presents quantification of the IL-1R positive area in cross-sections of aneurysms in vehicle and sham mice. FIG. 5C displays a bar graph representing localization of recombinant Anakinra to aneurysms of mice upon intravenous treatment with different concentrations (50 μg and 400 μg) of Ana-MB as assessed by ELISAs in indicated groups (n=4-5 per group). Healthy tissue was considered as negative control. Y-axis modified to account for differences in quantity. Data were analyzed by Student t test. FIG. 5D is a representative image of the increase in fluorescence in the aneurysms tissue, adjacent distal aorta and serum of the same mice depicting increase in localization of fluoresceine-labeled Anakinra conjugated methylene blue after tissues have been exposed to 600 nm light for fluorescence. FIG. 5E depicts quantification of the weekly percent growth in the maximal diameter of infrarenal abdominal aortas measured by micro-ultrasound imaging (MUI) using the B mode in indicated groups (n=10-15 per group). Measurements were performed weekly for 9 weeks following the induction of aneurysms. Treatment was administered on Day 21 post-induction of aneurysms. Data were analyzed by one-way ANOVA followed by the Kruskal Wallis post hoc test. *P<0.001. FIG. 5F shows representative images of abdominal aortas (white circles) visualized by MUI using the B mode in the indicated group. FIG. 5G displays representative images of the macroscopic features of AAA formation in indicated groups. FIG. 5H demonstrates quantification of the maximal diameter of infrarenal abdominal aortas measured by micrometry normalized to BW in indicated groups (n=10-15 per group). Data were analyzed by one-way ANOVA followed by the Kruskal Wallis post hoc test. AAA indicates abdominal aortic aneurysm; Ana-MB indicates methylene conjugated Anakinra; Ana-50, 50 μg of Anakinra-MB; Ana-400, 400 μg of Anakinra-MB; MB, methylene blue; Veh+MB+Light, photodynamic therapy; Veh+Ana-MB+Light, targeted photodynamic therapy.

FIGS. 6A, 6B and 6C show data demonstrating that MB PDT protects extracellular matrix from elastase-induced degradation. FIG. 6A shows representative image of porcine aortic cross-sections from indicated groups before and after treatment with elastase stained for elastin with Verhoeff-Van Gieson (VVG). FIG. 6B shows a bar graph of Ne-(carboxymethyl) lysine (CML) levels, a known marker of elastin crosslinking, in recombinant tropoelastin elastin treated with PDT and indicated controls. FIG. 6C shows a bar graph of CML levels in porcine tissue treated with PDT and indicated control groups, normalized to total elastin within tissue. AAA indicates abdominal aortic aneurysm; VVG staining, Verhoeff-Van staining; CML, Ne-(carboxymethyl) lysine; Veh, Vehicle (HBSS), Trop elastin, tropoelastin, PGG, Pentagalloyl glucose.

FIGS. 7A, 7B, and 7C show data demonstrating that photodynamic therapy improves mechanical strength of aortic tissue. FIG. 7A shows a representative image of healthy mouse aortic tissue subjected to treatment with vehicle, methylene blue without light, and with light (30 mW/cm²) at different timepoints (30 mins, 90 mins, and 120 mins). Glutaraldehyde treatment for 120 mins was used as a positive control. The relative change in stiffness was measured on a scale while holding the edge of the tissue with forceps. FIG. 7B shows a representative image of healthy porcine aortic tissue subjected to treatment with vehicle and with light for 120 mins at 60 mW/cm² and 120 mW/cm² intensity. Tissues were treated with recombinant elastase for 1 hour following light exposure to demonstrate resistance to elastin degradation. FIG. 7C shows the effect of MB PDT on stiffness of the aneurysm in aneurysms in vivo and the stiffness was assessed ex vivo (FIG. 7C).

FIG. 8 shows a schematic overview of an animal model for aortic aneurysms (AAA).

FIG. 9 shows show schematic overview of the treatment procedure.

### DETAILED DESCRIPTION

There is a need in the art for treating aneurysms. The present disclosure shows that using an activatable agent, e.g., a photosensitizer such as methylene blue can suppress vascular diameter growth, maintain stability of the vasculature, treat and/or stabilize an aneurysm, and/or increase extracellular matrix (ECM) crosslinking.

The present disclosure further provides for and exemplifies the non-invasive treatment of aneurysms by two means for selectively activating an activatable agent such as a photosensitizer at or near an aneurysm site in order to treat and/or stabilize an aneurysm. First, the activatable agent (e.g., a photosensitizer) contemplated herein is attached (e.g., conjugated) to a targeting moiety wherein the targeting moiety binds to an inflamed tissue. Aneurysms generally comprise inflammatory tissue. Inflammatory tissue preferentially expresses inflammatory markers, such as, IL1R. As such, in some embodiments, the targeting moiety binds IL1R. In one embodiment, the targeting moiety is anakinra. The ability to target a photosensitizer allows for systemic administration (e.g., via intravenous injection or infusion) and non-invasive treatment of the aneurysm. Second, an energy source (such as a light source coupled to a catheter) is used to activate the activatable agent via energy (e.g., by shining light) at or near the site of inflammation or the aneurysm to activate the photosensitizer locally. This can be beneficial because it provides an additional control for location specific activation by using light to activate the crosslinking reaction (e.g., restricting the therapeutic effect to the aneurysm). Additional disclosures as to the activatable agents (e.g., photosensitizers), activatable drug conjugates (e.g., photoactivatable drug conjugates), and methods for using the same are provided below.

### Activatable Agents

The activatable agents herein can be activated by any source of energy. In some embodiments, an activatable agent is a photosensitizer agent that is activatable by light. Photosensitizers (e.g., methylene blue) provided and described herein are generally advantageous in that they are activated when exposed to light at a wavelength within the tissue window (e.g., about 600 nm to about 1,000 nm), wherein activation reduces the rate of aneurysm growth; reduces an increase in aneurysm diameter, reduces/inhibits an increase in the volume of an aneurysm, inhibits aneurysm growth, promotes ECM crosslinking, and/or prevents the rupture of an aneurysm when activated at a site near and/or within an aneurysm. In addition to methylene blue, other photosensitizers are contemplated herein. In some embodiments, the photosensitizer is one that is activatable with light that can penetrate through blood or blood cells. In certain embodiments, the photosensitizer is one that generates singlet oxygen with exposed to light within the tissue window. In some instances, such range is 600 nm to about 1,000 nm. Other ranges near or overlapping with tissue window can be used, for example, 350 nm to 1,200 nm.

In some embodiments, the photosensitizer is a thiazine, porfimer sodium, a porphyrin, a chlorin, a pheophorbide, a phthalocyanine, an anthraquinone, a phenothiazine, a xanthene, or a cyanine, that generates singlet oxygen when exposed to light at a wavelength within the tissue window. In some embodiments, the photosensitizer is selected from Table 1.

TABLE 1

| Photosensitizer | PubChem CID | Excitation Wavelength | |
|---|---|---|---|
| Porfimer Sodium | 3086257 | ~630-690 | nm |
| Chlorin e6 (Ce6) | 5479494 | ~660-670 | nm |
| Pheophorbide a | 167186 | ~660-680 | nm |
| Purpurin-18 | 161493 | ~ 700-850 | nm |
| Aluminum Phthalocyanine | 114863 | ~660-690 | nm |
| Zinc Phthalocyanine | 114933 | ~650-680 | nm |
| Hypericin | 5281051 | around 600 | nm |
| Methylene Blue | 6099 | ~600-680 | nm |
| Rose Bengal | 32343 | ~500-550 | nm |
| indocyanine green | 5282412 | ~600-900 | nm |
| IR-780 | 118987185 | ~740-800 | nm |
| IR-820 | 53249358 | ~760-830 | nm |
| IR-783 | 53438012 | ~770-800 | nm |
| IRDye 700DX | 102004325 | ~675-700 | nm |
| IRDye 800CW | 168434453 | ~775 | nm |
| Verteporfin | 5362420 | ~689 | nm |
| Methylene Purple | 73024 | ~640-660 | nm |

In some embodiments, the photosensitizer is hematoporphyrins, naturally occurring bacteriochlorins, pheophorbides, pyropheophorbide-a, photochloride, chlorins, chlorin e6, mono-L-aspartyl 10 chlorin e6, di-L-aspartyl chlorin e6, tin (IV) chlorin e6, palladium bacteriochlorophylls, palladium bacteriopheophorbides, synthetic chlorins, synthetic bacteriochlorins, metatetrahydroxyphenyl chlorin, a bacteriopheophorbide, bacteriochlorin, benzoporphyrin, monobenzoporphyrins, verteporfin, sulfonated aluminum phthalocyanines (disulfonated and tetrasulfonated), sulfonated aluminum naphthalocyanines, tin and zinc octaethylpurpurine, tin ethiopurpurine, vegetables porficenos, synthetic porphyrins, chlorins synthetic, synthetic bacteriochlorins, mesotrietinylporphyrins without metals, metallized mesotrietinylporphyrins, modified core porphyrins, expanded porphyrins (texaphyrins), tocopherol, Lutetium motexaphine, gadolinium motexafin, merocyanine 540, acridine dyes, hypericin, halogenated squarin dyes, halogenated xanthene dyes, eosin, talaporfin sodium, 2-(1-Hexyloxyethyl)-2-Devinyl Pyropheophorbide-a (HPPH-Photochlor), benzo-porphyrin derivative monoacid ring A, redaporfin (LUZ11), chlorin E6/P6/purpurin, Ru(II) polypyridyl complex (TLD-1433), padeliporfin di-potassium, or 5-aminolevulinic acid hydrochloride.

### Targeting Moieties

As described and exemplified herein, activatable agent(s) (e.g., photosensitizer(s)) can be attached (coupled/conjugated) to a targeting moiety in order to target the activatable agent(s) to a site near and/or within an aneurysm, concentrate the activatable agent(s) at the site of near and/or within an aneurysm, and/or reduce the overall exposure of the subject to the activatable agent(s). A targeting moiety is preferably one that targets one or more photosensitizers to an inflammatory tissue (e.g., by binding an inflammation-associated cell surface receptor). Such inflammatory tissue can be inflammatory tissue of the vasculature, such as at a site near or of an aneurysm. In some embodiments, the targeting moiety does not selectively bind to a cancer or tumor cell. Alternatively, the targeting moiety can be one that selectively binds non-malignant cells.

Anakinra is a targeting moiety that binds an inflammatory (e.g., inflammation associated) cell surface receptor IL1R. In some embodiments, Anakinra is conjugated to an activatable agent(s). For example, activatable drug conjugates (ADCs) having anakinra attached to one or more methylene blue photosensitizers (e.g., through an amide linkage) demonstrate the ability to target the photosensitizer(s) to a site near and/or within an aneurysm, concentrate the photosensitizer(s) at the site near and/or within an aneurysm, and/or reduce the overall exposure of the subject to the photosensitizer(s). In turn, activating a photosensitizer such as a methylene blue photosensitizer at a site near or within an aneurysm (e.g., via an optical catheter) reduces an increase in aneurysm diameter; reduces an increase in the volume of an aneurysm, increases ECM crosslinking and/or prevents rupture of an aneurysm.

In some embodiments, an activatable agent, such as a photosensitizer is attached to a targeting moiety other than anakinra, or to a targeting moiety that targets a marker other than IL1R. In such embodiments a targeting moiety can selectively target an inflammatory (e.g., inflammation associated) cell surface receptor such as any one of: CCR1, IL1R, IFNAR1, TNF-R2, CXCR2, CCR2, IL2R, IFNAR2, CD40, CXCR3, CCR3, IL3R, IFNGR1, CD30, CXCR4, CCR4, IL4R, IFNGR2, CD27, CXCR5, CCR5, IL5R, IFNLR1, CD28, CXCR6, CCR6, IL6R, CD95, CXCR7, CCR7, IL7R, LTbR, CXC3R1, CCR8, IL8R, OX40, CCR9, IL9R, 4-1BB, CCR10, IL10R, BAFFR, IL11R, BCMA, IL12R, TACI, IL13R, RANK, IL15R, NGFR, IL17R, TROY, IL18R, EDAR, IL20R, RELT, IL21R, Fn14, IL22R, TIM3, IL23R, IL27R, IL28R, or IL31R.

Such targeting moiety can be an antibody, an antibody fragment, a protein or peptide, a small molecule, a ligand, etc. In certain embodiments, antibody fragments described herein comprises a single chain variable fragment (scFv), a Fab, Fab2, Fab3, F(ab′)2 diabody, triabody, tetrabody, BiTE, TandAB or DART.

In some embodiments, the targeting moiety comprises anakinra. In certain embodiments, the targeting moiety comprises SEQ ID NO: 1 or an amino acid sequence having at least 85%, 90%, 95%, 98%, or 99% sequence identity to SEQ ID NO: 1, and binds IL1R. In some embodiments, the targeting moiety comprises EBI-005. In certain embodiments, the targeting moiety comprises SEQ ID NO: 2 or 3 or an amino acid sequence having at least 85%, 90%, 95%, 98%, or 99% sequence identity to SEQ ID NO: 2 or 3 and binds IL1R. In some embodiments, the targeting moiety comprises Rilonacept. In certain embodiments, the targeting moiety comprises SEQ ID NO: 4 or an amino acid sequence having at least 85%, 90%, 95%, 98%, or 99% sequence identity to SEQ ID NO: 4, and binds IL1-beta.

In some embodiments, the targeting moiety is the antibody Nadunolimab. In certain embodiments, the targeting moiety is an antibody comprising a VH domain comprising SEQ ID NO: 9 and a VL domain comprising SEQ ID NO: 10, or an amino acid sequence having at least 85%, 90%, 95%, 98%, or 99% sequence identity to SEQ ID NO: 9 and/or 10, and binds IL1R. In certain embodiments, the targeting moiety is CDX-1140 targeting CD40. In certain embodiments, the targeting moiety is AFM13 targeting CD30. In certain embodiments, the targeting moiety is SEA-CD30 targeting CD30. In certain embodiments, the targeting moiety is KW-0761 targeting C—C chemokine receptor 4 (CCR4). In certain embodiments, the targeting moiety is AMG 317 targeting Interleukin-4 receptor (IL4R). In certain embodiments, the targeting moiety is Olokizumab targeting Interleukin-6 receptor (IL6R). In certain embodiments, the targeting moiety is MEDI6383 targeting OX40. In certain embodiments, the targeting moiety is PF-05082566 targeting 4-1BB. In certain embodiments, the targeting moiety is AMG 139 targeting Interleukin-10 receptor (IL10R). In certain embodiments, the targeting moiety is GSK2857916 targeting B-cell activating factor receptor (BAFFR). In certain embodiments, the targeting moiety is Teclistamab targeting B-cell maturation antigen (BCMA). In certain embodiments, the targeting moiety is CC-92480 targeting B-cell maturation antigen (BCMA). In certain embodiments, the targeting moiety is JNJ-64007957 targeting B-cell maturation antigen (BCMA). In certain embodiments, the targeting moiety is Blisibimod targeting Transmembrane activator and CAML interactor (TACI). In certain embodiments, the targeting moiety is Atacicept targeting Transmembrane activator and CAML interactor (TACI). In certain embodiments, the targeting moiety is Tralokinumab targeting Interleukin-13 receptor (IL13R). In certain embodiments, the targeting moiety is Secukinumab targeting Interleukin-17 receptor (IL17R). In certain embodiments, the targeting moiety is Ixekizumab targeting Interleukin-17 receptor (IL17R). In certain embodiments, the targeting moiety is Sabatolimab targeting T-cell immunoglobulin and mucin domain 3 (TIM3). In certain embodiments, the targeting moiety is TSR-022 targeting T-cell immunoglobulin and mucin domain 3 (TIM3). In certain embodiments, the targeting moiety is Ustekinumab targeting Interleukin-23 receptor (IL23R). In some embodiments, the targeting moiety is the antibody canakinumab. In certain embodiments, the targeting moiety is an antibody comprising a VH domain comprising SEQ ID NO: 5 and a VL domain comprising SEQ ID NO: 6, or an amino acid sequence having at least 85%, 90%, 95%, 98%, or 99% sequence identity to SEQ ID NO: 5 and/or 6, and binds IL1-beta. In some embodiments, the targeting moiety is the antibody gevokizumab. In certain embodiments, the targeting moiety is an antibody comprising a VH domain comprising SEQ ID NO: 7 and a VL domain comprising SEQ ID NO: 8, or an amino acid sequence having at least 85%, 90%, 95%, 98%, or 99% sequence identity to SEQ ID NO: 7 and/or 8, wherein the antibody binds IL1-beta. In certain embodiments, the targeting moiety is LY2189102 targeting IL1-beta.

### Activatable Drug Conjugates

Any of the activatable agents (e.g., photosensitizers) herein can be coupled to a targeting moiety using known conjugation technologies to form an activatable drug conjugate (ADC). Conjugation can optionally include the addition of one or more spacers or linkers into the ADC. Techniques for conjugation of an activatable agent such as a photosensitizer to a targeting moiety contemplated herein include, for example: ThermoFisher. Bioconjugation and crosslinking technical handbook: Reagents for bioconjugation, crosslinking, biotinylation, and modification of proteins and peptides. 2022; Bioconjugation: Methods and Protocols. 22 Jul. 2019. ISBN: 978-1-4939-9654-4; and Khongorzul P, Ling C J, Khan F U, Ihsan A U, Zhang J. Antibody-Drug Conjugates: A Comprehensive Review. Mol Cancer Res. 2020 January; 18(1):3-19. Epub 2019 Oct. 28. PMID: 31659006; Su Z, Xiao D, Xie F, Liu L, Wang Y, Fan S, Zhou X, Li S. Antibody-drug conjugates: Recent advances in linker chemistry. Acta Pharm Sin B. 2021 December; 11(12):3889-3907. Epub 2021 Apr. 6. PMID: 35024314; PMCID: PMC8727783, and Tong J T W, Harris P W R, Brimble M A, Kavianinia I. An Insight into FDA Approved Antibody-Drug Conjugates for Cancer Therapy. Molecules. 2021 Sep. 27; 26(19):5847. PMID: 34641391.

By way of example, anakinra is conjugated to one or more methylene blue photosensitizers via an amide linkage formed by reacting the methylene blue NHS ester and one or more lysine residues on anakinra following the protocol provided in Example 4. Methylene blue NHS ester comprises a structure represented by the formula:

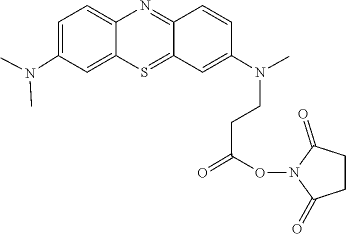

In one embodiment, anakinra is conjugated to one or more methylene blue photosensitizers via an amide linkage formed by reacting the methylene blue NHS ester and one or more lysine residues on anakinra.

One or more activatable agents or photosensitizers can be attached to the targeting moiety through a linkage comprising an amide linkage, an oxime linkage, triazole linkage, or a thioether linkage. Anakinra Conjugated forms of MB include a structure represented by the formula:

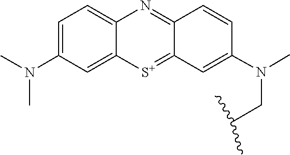

Techniques for making methylene blue and methylene blue derivatives are contemplated herein include, for example: Khadieva A, Rayanov M, Shibaeva K, Piskunov A, Padnya P, Stoikov I. Towards Asymmetrical Methylene Blue Analogues: Synthesis and Reactivity of 3-N′-Arylaminophenothiazines. Molecules. 2022 May 8; 27(9):3024. doi: 10.3390/molecules27093024. PMID: 35566375.

In certain embodiments, the linkage further introduces a spacer between the linker and the photosensitizer. In some embodiments, the spacer is a heteroatom. In some embodiments, the spacer is an alkyl chain. In some embodiments, the spacer is an alkyl chain comprising one or more heteroatoms. In some embodiments, the spacer is a carbonyl group. In certain embodiments, the photosensitizer is directedly attached to the targeting moiety.

A linker (e.g., bifunctional linkage) can also be introduced via a linkage. In some embodiments, the linkage introduces a linker, wherein the photosensitizer is further attached to the targeting moiety via the linker. In some embodiments, a linker or a linker precursor used to form the linker comprises 6-maleimidocaproyl (MC), Maleimide-DOTA, maleimidopropanoyl (MP), alanine-phenylalanine (AP), p-aminobenzyloxycarbonyl (PAB), N-succinimidyl 4-(2-pyridylthio) pentanoate (SPP), N-succinimidyl 4-(N-maleimidomethyl)cyclohexane-1-carboxylate (SMCC), N-succinimidyl (4-iodo-acetyl)aminobenzoate (SIAB), valine-citrulline (VC), 6-maleimidocaproyl-valine-citrulline (MC-VC), 6-maleimidocaproyl-valine-citrulline-p-aminobenzyloxycarbonyl (MC-VC-PAB), N-succinimidyl-1-carboxylate-valine-citrulline-p-aminobenzyloxy-carbonyl (SC-VC-PAB), 6-maleimidocaproyl-polyethylene glycol-valine-citrulline (MC-PEG4-VC), 6-maleimidocaproyl-polyethylene glycol-valine-alanine (MC-PEG4-VA), or MC-PEG8-VC-PAB. In certain embodiments, the linker precursor is 6-maleimidocaproyl-valine-citrulline-p-aminobenzyloxycarbonyl (MC-VC-PAB), or N-succinimidyl-1-carboxylate-valine-citrulline-p-aminobenzyloxycarbonyl (SC-VC-PAB).

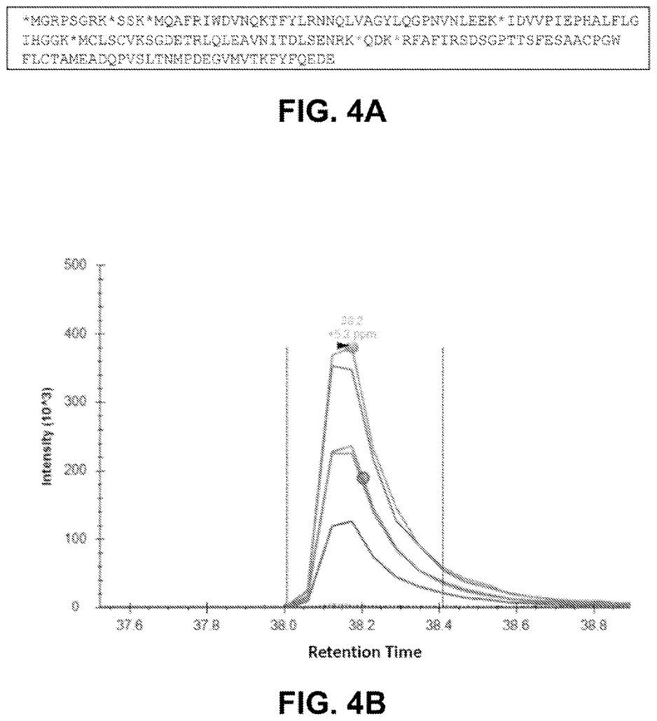

In certain embodiments, the linkers described herein may be attached to the targeting moieties described herein at a naturally occurring amino acid residue such as a lysine or a reduced cysteine. In certain embodiments, the photosensitizer is attached to a cysteine residue of the targeting moiety. In some embodiments, the photosensitizer is attached to a lysine residue of the targeting moiety. In certain embodiments, the linkers described herein may be attached to the targeting moieties described herein at a non-naturally occurring amino acid residue introduced into a protein sequence. In some embodiments, the one or more lysine residues is at positions K7, K10, K46, K65, K94, and K97 of FIG. 4A of SEQ ID NO: 1.

As such, in some embodiments, the ADC comprises a heterogenous composition of one or more activatable agents (e.g., photosensitizers) conjugated to the targeting moieties. For example, in certain embodiments, the ADC comprises a heterogenous composition of one or more methylene blue photosensitizers conjugated to the anakinra.

In certain embodiments, the linker is first conjugated to the photosensitizer, and then subsequently conjugated to the targeting moiety. In certain embodiments, the linker is first conjugated to the targeting moiety, and then subsequently conjugated to the photosensitizer.

In some embodiments, provided herein are pharmaceutical compositions and/or formulations comprising the photosensitizer or the activatable drug conjugate. Pharmaceutical compositions include and/or refers a preparation that is in such form as to permit the biological activity of the photosensitizer alone or in the conjugate to be effective, and that contains no additional components which are unacceptably toxic to a subject to which the formulation would be administered. In certain embodiments, the pharmaceutical composition comprises a pharmaceutically acceptable carrier (e.g., an ingredient in a pharmaceutical formulation, other than an active ingredient, which is nontoxic to an individual). A pharmaceutically acceptable carrier includes, but is not limited to, a buffer, excipient, stabilizer, or preservative.

### Systems

Provided and described herein are systems useful for treating an aneurysm. In some embodiments, a system comprises: an activatable drug conjugate and an energy source to activate the agent. For example, as described herein, the activatable drug conjugate can be a photoactivatable drug conjugate (e.g., comprising anakinra attached to methylene blue) and an energy source that delivers light delivery unit such as an optical applicator. Such a system can also comprise a light generating unit comprising a light source. In certain embodiments, the optical applicator comprises a catheter configured to deliver light at the range required to activate the photosensitizer incorporated in the system. For example, a system comprising an anakinra-methylene blue conjugate comprises a light source configured to emit light in the range of 600 nm to 1000 nm. Preferably, the catheter is configured for delivery of light within a blood vessel at the location of or near the site of an aneurysm.

In some instances, the catheter is a laser catheter and/or light-emitting diode catheter. In certain embodiments, the catheter contains optical fibers to deliver the light. In certain embodiments, the light delivery unit is attached to the light generator and facilitates the transmission of light (e.g., a laser beam).

In certain embodiments, the light generating unit and/or the light delivery unit is configured to administer light at an irradiance of about 1 mW/cm² to about 600 mW/cm². In certain embodiments, the light generating unit and/or the light delivery unit is configured to administer light at an irradiance of about 25 mW/cm² to about 100 mW/cm².

In some embodiments, also provided and described herein are kits comprising one or more components of the systems described herein. For example, a kit can include all components of the photodynamic therapy systems, or only the photoactivatable drug conjugate (e.g., any one of the photoactivatable drug conjugates described herein) and a light delivery unit.

### Methods

### Methods for Treating Aneurysms

Provided and describe herein are methods for treating an aneurysm. In some embodiments, the methods comprise:
(a) administering to a subject in need thereof an activatable agent; and
(b) activating the activatable agent at the site of or near the aneurysm using light, wherein activating the activatable agent comprises delivering energy to the site or near the site of the aneurysm. In some embodiments, the methods comprise:
(a) administering a activatable drug conjugate (e.g., anakinra linked to one or more methylene blue photosensitizers); and (b) activating the activatable agent at the site of or near the aneurysm using light, wherein activating the activatable agent comprises delivering energy to the site or near the site of the aneurysm.

Also provided and described herein are, in certain embodiments, methods advantageous for decelerating aneurysm progression, reducing elastase-induce extracellular matrix degradation, increasing ECM crosslinking, and/or improving mechanical strength of aortic tissue. In some embodiments, the methods comprising: (a) administering an activatable agent (e.g., a photosensitizer); and (b) activating the activatable agent at the site of or near the aneurysm using light, wherein activating the activatable agent comprises delivering energy to the site or near the site of the aneurysm. In some embodiments, the methods comprise: (a) administering a activatable drug conjugate (e.g., anakinra linked to one or more methylene blue photosensitizers); and (b) activating the activatable agent at the site of or near the aneurysm using light, wherein activating the activatable agent comprises delivering energy to the site or near the site of the aneurysm

In some embodiments, the activatable drug conjugate localizes to regions near or within tissue inflammation or an aneurysm, as compared to a region of healthy tissue. In some embodiments, the activatable agent is a photosensitizer and activating the photosensitizer comprises photoactivation.

As described and exemplified herein, in some embodiments, activating the activatable agent (e.g., photoactivating the photosensitizer): (i) reduces or maintains the diameter of a blood vessel having an aneurysm, as compared to an untreated blood vessel having an aneurysm; (ii) decreases the volume of an aneurysm or inhibits an increase in the volume of the aneurysm, as compared to an untreated aneurysm; (iii) reduces the rate of blood vessel diameter growth in an blood vessel having an aneurysm, as compared to an untreated blood vessel having an aneurysm; and/or (iv) decreases the rate of volume increase in an aneurysm or inhibits an increase in the volume of the aneurysm, as compared to an untreated aneurysm.

The methods described herein can be used for aneurysms within different anatomical regions. In some embodiments, the aneurysm is an aortic aneurysm, a cerebral aneurysm, a thoracic aortic aneurysm, an abdominal aortic aneurysm, a popliteal artery aneurysm, a peripheral aneurysm, a fusiform aneurysm, a saccular aneurysm, or a mycotic aneurysm. In certain embodiments, the aneurysm is an aortic aneurysm In certain embodiments, the aneurysm is a cerebral aneurysm. In certain embodiments, the aneurysm is a thoracic aortic aneurysm. In certain embodiments, the aneurysm is an abdominal aortic aneurysm. In certain embodiments, the aneurysm is a popliteal artery aneurysm. In certain embodiments, the aneurysm is a peripheral aneurysm. In certain embodiments, the aneurysm is a fusiform aneurysm. In certain embodiments, the aneurysm is a saccular aneurysm, a mycotic aneurysm. In certain embodiments, the aneurysm is a pseudo aneurysm. In certain embodiments, the blood vessel is the aorta.

In certain embodiments, photoactivating takes pace within an individual, e.g., in (b), through the use of an optical applicator. In certain embodiments, the optical applicator comprises a catheter configured to deliver light (e.g., a laser catheter and/or light-emitting diode catheter). In certain embodiments, the catheter contains optical fibers to deliver the light. In certain embodiments, the optical applicator (e.g., catheter) is inserted through an artery. In certain embodiments, the optical applicator (e.g., catheter) is inserted through the groin artery.

The photoactivatable drug conjugate can be administered by any suitable means, including parenteral, and, if desired for local treatment. Parenteral infusions include intravenous and intraarterial administration. Dosing can be by any suitable route. Various dosing schedules can be implemented, including but not limited to single or multiple administrations over various time-points, bolus administration, and pulse infusion. In certain embodiments, the photoactivatable drug conjugate is administered via parenteral infusion or injection. In certain embodiments, the photoactivatable drug conjugate is administered via intravenous infusion or injection.

The methods herein are also useful for extending the therapeutic window for treating an aneurysm. In some embodiments, administering the photosensitizer or photoactivatable drug conjugate and activating the photosensitizer can allow for delayed surgical intervention for an aneurysm due to reduced long term growth rate.

In certain embodiments, the aneurysm is an abdominal aortic aneurysm. In certain embodiments, the aneurysm is a cerebral aneurysm. In certain embodiments, the aneurysm is a thoracic aortic aneurysm. In certain embodiments, the aneurysm is an abdominal aortic aneurysm. In certain embodiments, the aneurysm is a popliteal artery aneurysm. In certain embodiments, the aneurysm is a peripheral aneurysm. In certain embodiments, the aneurysm is a fusiform aneurysm. In certain embodiments, the aneurysm is a saccular aneurysm. In certain embodiments, the aneurysm is a mycotic aneurysm. In certain embodiments, the aneurysm is a pseudo aneurysm.

In certain embodiments, the dose of the photosensitizer is less than 4 per dose. In certain instances, the targetable moiety enables a therapeutic effect (e.g., increasing an amount of tissue) at lower amounts of the activatable agent or photosensitizer that if administered without the targeting moiety (i.e., in a non-conjugate manner).

In certain embodiments, the light is administered by a laser catheter. In certain embodiments, the light is administered at an irradiance of about 1 mW/cm² to about 600 mW/cm². In certain embodiments, the light is administered at an irradiance of about 25 mW/cm² to about 100 mW/cm².

### Definitions

As used herein, percent (%) sequence identity or “sequence identity”, and terms related thereto, in the context of amino acid sequences or nucleic acid sequences, generally refer to and include the percentage of amino acid residues or nucleic acid residues in a candidate sequence that are identical with the amino acid residues or nucleic acid residues, respectively, in a selected sequence, after aligning the sequences and introducing gaps, if necessary, to achieve the maximum percent sequence identity, and not considering any conservative substitutions as part of the sequence identity. Alignment for purposes of determining percent amino acid sequence identity or percent nucleic acid identity can be achieved in various ways that are within the skill in the art, for instance, using publicly available computer software such as Clustal Omega, BLAST, BLAST-2, ALIGN, ALIGN-2 or Megalign (DNASTAR) software, with BLAST being the alignment algorithm of preference. Those skilled in the art can determine appropriate parameters for measuring alignment, including any algorithms needed to achieve maximal alignment over the full-length of the sequences being compared, although for simplicity it maybe preferred to use default parameters.

The term “antibody” has used herein is used in the broadest sense and generally refers to and/or includes monoclonal antibodies, multi-valent antibodies, multi-specific, antigen-binding fragments of antibodies. Antigen-binding fragments of antibodies (antibody fragments) generally refer to and/or include antibody-derived proteins that comprise a functional set of CDRs (e.g., a CDR-H1-3 and CDR-L1-3) that bind a target protein and have a molecule weight less than a full length IgG antibody (e.g., a molecular weight less than ~150,000 Daltons). In certain embodiments, an antigen-binding antibody fragment includes: fragment antigen binding (Fab) fragments, F(ab′)2 fragments, Fab′ fragments, Fv fragments, IgG (rIgG) fragments, and single chain antibody fragments, including single chain variable fragments (sFv or scFv). Antibodies and antigen-binding fragments of antibodies generally encompass genetically engineered, and/or otherwise modified forms of immunoglobulins, such as intrabodies, peptibodies, chimeric antibodies, fully human antibodies, humanized antibodies, and heteroconjugate antibodies, multi-specific antibodies, multi-valent antibodies, diabodies, triabodies, and tetrabodies, tandem di-scFv, tandem tri-scFv. A full-length antibody or antibody fragment, intact antibody or antibody fragment, and/or whole antibody are interchangeable, and generally include and/or refer to an antibody having a structure substantially similar to a native antibody structure having heavy chains that contain an Fc region and/or include antibodies of any class or sub-class, including IgG and sub-classes thereof (e.g., IgG1 and IgG4), IgM, IgE, IgA, and IgD.

“Complementarity determining regions” (CDRs) as used herein are synonymous with “hypervariable regions” (HVRs) generally refer to and/or include non-contiguous sequences of amino acids within antibody variable regions that confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). Framework regions (FRs) generally refer to and/or include non-CDR regions of the heavy and light chain variable regions. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). Variable regions (also referred to as variable domains) generally refer to and/or include the domain of an antibody heavy or light chain that is involved in binding the antibody to antigen (e.g., a single variable domain comprises a CDR 1, CDR 2, and CDR 3). The variable domains of the heavy chain and light chain (VH and VL, respectively) of a native antibody generally have similar structures, with each domain comprising four conserved framework regions (FRs) and three CDRs In certain instances, a single VH or VL domain can be sufficient to confer antigen-binding. Fc region generally encompasses and/or refers to a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991. In certain embodiments, the Fc region include IgG and sub-classes thereof (e.g., IgG1 and IgG4), IgM, IgE, IgA, and/or IgD heavy chain constant regions and/or heavy chain constant regions derived from IgG and sub-classes thereof (e.g., IgG1 and IgG4), IgM, IgE, IgA, and IgD.

An “individual” as used herein is synonymous with “patient” and/or “subject” and includes and/or refers to a human and may be a human that has been diagnosed as needing to treat a disease or condition as disclosed herein. However, examples are not limited to humans and include, chimpanzees, marmosets, cows, horses, sheep, goats, pigs, rabbits, dogs, cats, rats, mice, guinea pigs, and the like. The individual is typically a human and may be a human that has been diagnosed as needing to treat a disease or condition as disclosed herein.

“Treating” or “treatment” as herein includes and/or refers to ameliorating the disease or disorder or symptoms thereof (e.g., slowing or arresting or reducing the development of the disease or at least one of the clinical symptoms thereof). In certain embodiments, treating or treatment also includes and/or refers to alleviating or ameliorating at least one physical and/or biological parameters including those which may not be discernible by the patient. In certain embodiments, treating or treatment includes and/or refers to modulating a disease, disorder, or biological process either physically, (e.g., stabilization of a discernible symptom), physiologically, (e.g., stabilization of a physical and/or biological parameter), or both. In certain embodiments, treating or treatment includes and/or refers to preventing or delaying the onset or development or progression of the disease or disorder. In certain embodiments, treating or treatment includes and/or refers to preventing or delaying or inhibiting the deterioration of (i) a healthy physiological state or (ii) a baseline physiological state (e.g., the progression of a disease or disorder).

As used herein, in any instance or embodiment described herein, “comprising” may be replaced with “consisting essentially of” and/or “consisting of,” unless context clearly connotes otherwise. Similarly, as used herein, in any instance or embodiment described herein, “comprises” may be replaced with “consists essentially of” and/or “consists of,” unless context clearly connotes otherwise.

As used herein, the term “about,” in the context of a given value or range, includes and/or refers to a value or range that is within 10% of the given value or range.

As used herein, the term “and/or” is to be taken as specific disclosure of each of the two specified features or components with or without the other. For example, “A and/or B” is to be taken as specific disclosure of each of (i) A, (ii) B and (iii) A and B, just as if each were set out individually herein.

### EXEMPLARY EMBODIMENTS

Exemplary embodiment 1. A photoactivatable drug conjugate comprising anakinra attached to a photosensitizer is activated when exposed to light at a wavelength of about 600 nm to about 1000 nm.

Exemplary embodiment 2. The photoactivatable drug conjugate of embodiment 1, wherein photoactivation of the photosensitizer:
(i) reduces the rate of aneurysm growth and/or reduces/maintains the diameter of a blood vessel having an aneurysm, as compared to an untreated blood vessel having an aneurysm;
(ii) decreases the volume of an aneurysm or inhibits an increase in the volume of the aneurysm, as compared to an untreated aneurysm;
(iii) reduces the rate of blood vessel diameter growth in an blood vessel having an aneurysm, as compared to an untreated blood vessel having an aneurysm; and/or
(iv) decreases the rate of volume increase in an aneurysm or inhibits an increase in the volume of the aneurysm, as compared to an untreated aneurysm.

Exemplary embodiment 3. The photoactivatable drug conjugate of any one of embodiments 1-2, wherein the photoactivatable drug conjugate localizes to regions near or within tissue inflammation or an aneurysm, as compared to a region of healthy tissue.

Exemplary embodiment 4. The photoactivatable drug conjugate of any one of embodiments 1-3, wherein the photosensitizer is selected from: porfimer sodium, a porphyrin, a chlorin, a pheophorbide, a bacteriopheophorbide, a phthalocyanine, a anthraquinone, a phenothiazine, a xanthene, or a cyanine, optionally wherein the photosensitize is elected from Table 1.

Exemplary embodiment 5. The photoactivatable drug conjugate of any one of embodiments 1-4, wherein the photosensitizer generates a singlet oxygen when exposed to light at a wavelength of about 600 nm to about 1000 nm.

Exemplary embodiment 6. A photoactivatable drug conjugate comprising anakinra attached via a linker to methylene blue.

Exemplary embodiment 7. The photoactivatable drug conjugate of any one of embodiments 1-6, wherein the anakinra comprises an amino acid sequence having at least 85%, 90%, 95%, 97%, 98%, 99%, or greater sequence identity to SEQ ID NO: 1, wherein the anakinra binds IL1R.

Exemplary embodiment 8. The photoactivatable drug conjugate of embodiment 7, wherein the anakinra comprises an amino acid sequence of SEQ ID NO: 1.

Exemplary embodiment 9. The photoactivatable drug conjugate of any one of embodiments 1-8, wherein the photosensitizer is covalently attached to the anakinra.

Exemplary embodiment 10. The photoactivatable drug conjugate of any one of embodiments 1-9, wherein the photosensitizer is attached to the anakinra via a linker.

Exemplary embodiment 11. The photoactivatable drug conjugate of any one of embodiments 1-10, wherein the photosensitizer is attached to the targeting moiety via a linkage comprising an amide linkage, an oxime linkage, triazole linkage, or a thioether linkage.

Exemplary embodiment 12. The photoactivatable drug conjugate of any one of embodiments 1-11, wherein the photosensitizer is attached to one or more lysine residues or cysteine residues of the anakinra.

Exemplary embodiment 13. The photoactivatable drug conjugate of any one of embodiments 1-11, wherein the photoactivatable drug conjugate comprises a plurality of photosensitizers (e.g., methylene blue) attached the anakinra.

Exemplary embodiment 14. A photoactivatable drug conjugate comprising a photosensitizer attached to a targeting moiety, wherein the targeting moiety binds an inflamed tissue.

Exemplary embodiment 15. The photoactivatable drug conjugate of embodiment 14, wherein the inflamed tissue is a vascular tissue.

Exemplary embodiment 16. The photoactivatable drug conjugate any one of embodiments 14-15, wherein the inflamed tissue is a blood vessel.

Exemplary embodiment 17. The photoactivatable drug conjugate any one of embodiments 14-16, wherein the inflamed tissue is within or near the site of an aneurysm.

Exemplary embodiment 18. The photoactivatable drug conjugate any one of embodiments 14-17, wherein the targeting moiety binds to a cytokine receptor or an inflammatory molecule.

Exemplary embodiment 19. The photoactivatable drug conjugate of any one of embodiments 14-18, wherein photoactivation of the photosensitizer:
(i) reduces the rate of aneurysms growth and/or reduces/maintains the diameter a blood vessel having an aneurysm, as compared to an untreated blood vessel having an aneurysm;
(ii) decreases the volume of an aneurysm or reduces rate of increase in the volume of the aneurysm, as compared to an untreated aneurysm;
(iii) reduces the rate of blood vessel diameter growth in an blood vessel having an aneurysm, as compared to an untreated blood vessel having an aneurysm; and/or
(iv) decreases the rate of volume increase in an aneurysm or inhibits an increase in the volume of the aneurysm, as compared to an untreated aneurysm.

Exemplary embodiment 20. The photoactivatable drug conjugate of any one of embodiments 14-19, wherein the photoactivatable drug conjugate localizes to regions near or within tissue inflammation or an aneurysm, as compared to a region of healthy tissue.

Exemplary embodiment 21. The photoactivatable drug conjugate of any one of embodiments 14-20, wherein the photosensitizer generates singlet oxygen when exposed to light at a wavelength of about 350 nm to about 1200 nm.

Exemplary embodiment 22. The photoactivatable drug conjugate of any one of embodiments 14-21, wherein the photosensitizer is activated when exposed to light at a wavelength of about 600 nm to about 1000 nm.

Exemplary embodiment 23. The photoactivatable drug conjugate of any one of embodiments 14-22, wherein the photosensitizer is selected from: porfimer sodium, a porphyrin, a chlorin, a pheophorbide, a bacteriopheophorbide, a phthalocyanine, a anthraquinone, a phenothiazine, a xanthene, or a cyanine, or optionally, the photosensitizer is selected from Table 1.

Exemplary embodiment 24. The photoactivatable drug conjugate of any one of embodiments 14-23, wherein the photosensitizer is methylene blue.

Exemplary embodiment 25. The photoactivatable drug conjugate of any one of embodiments 14-23, wherein the photosensitizer generates singlet oxygen when photoactivated.

Exemplary embodiment 26. The photoactivatable drug conjugate of any one of embodiments 14-25, wherein the photosensitizer is covalently attached to the targeting moiety.

Exemplary embodiment 27. The photoactivatable drug conjugate of any one of embodiments 14-26, wherein the photosensitizer is attached to the targeting moiety via a linker.

Exemplary embodiment 28. The photoactivatable drug conjugate of any one of embodiments 13-23, wherein the photosensitizer is attached to the targeting moiety via a linkage comprising an amide linkage, an oxime linkage, triazole linkage, or a thioether linkage.

Exemplary embodiment 29. The photoactivatable drug conjugate of any one of embodiments 14-28, wherein the photosensitizer is attached to one or more cysteine residues or lysine residues of the targeting moiety.

Exemplary embodiment 30. The photoactivatable drug conjugate of any one of embodiments 14-29, wherein the photoactivatable drug conjugate comprises a plurality of photosensitizers attached to the targeting moiety.

Exemplary embodiment 31. The photoactivatable drug conjugate of any one of embodiments 14-30, wherein the targeting moiety is selected from: an antibody or antibody fragment, a protein, a ligand, or a small molecule.

Exemplary embodiment 32. The photoactivatable drug conjugate of any one of embodiments 14-31, wherein the targeting moiety binds IL1R.

Exemplary embodiment 33. The photoactivatable drug conjugate of any one of embodiments 14-32, wherein the targeting moiety comprises an amino acid sequence having at least 90% sequence identity to any one of SEQ ID NOs: 1-3, wherein the targeting moiety binds IL1R.

Exemplary embodiment 34. The photoactivatable drug conjugate of any one of embodiments 14-33, wherein the targeting moiety comprises an amino acid sequence of any one of SEQ ID NOs: 1-10.

Exemplary embodiment 35. The photoactivatable drug conjugate of any one of embodiments 14-34, wherein the targeting moiety binds IL1beta.

Exemplary embodiment 36. The photoactivatable drug conjugate of embodiment 35, wherein the targeting moiety is an antibody comprising a heavy chain variable domain having at least 90% sequence identity to SEQ ID NO: 5 and a light chain variable domain having at least 90% sequence identity to SEQ ID NO: 6.

Exemplary embodiment 37. The photoactivatable drug conjugate of embodiment 35, wherein the targeting moiety is an antibody comprising a heavy chain variable domain having at least 90% sequence identity to SEQ ID NO: 7 and a light chain variable domain having at least 90% sequence identity to SEQ ID NO: 8.

Exemplary embodiment 38. A pharmaceutical composition comprising the photoactivatable drug conjugate of any one of embodiments 1-37 and a pharmaceutically acceptable excipient.

Exemplary embodiment 39. A system comprising: the photoactivatable drug conjugate of any one of embodiments 1-37; and a light delivery unit.

Exemplary embodiment 40. The system of embodiment 39, wherein the light delivery unit comprises an optical applicator.

Exemplary embodiment 41. The system of embodiment 40, wherein the optical applicator comprises a catheter configured to deliver light.

Exemplary embodiment 42. The system of embodiment 41, wherein the catheter contains optical fibers to deliver the light.

Exemplary embodiment 43. The system of any one of embodiments 39-42, wherein the light generating unit generates light having a wavelength of about 600 nm to about 1000 nm.

Exemplary embodiment 44. The system of any one of embodiments 39-43, wherein the light delivery unit is capable of delivering light to tissue of an individual.

Exemplary embodiment 45. The system of embodiment 44, wherein the tissue is within or near the site of an aneurysm.

Exemplary embodiment 46. The system of embodiments 44, wherein the light delivery unit is capable of delivering light a blood vessel.

Exemplary embodiment 47. The system of embodiments 44, wherein the light delivery unit is capable of delivering light to the aorta.

Exemplary embodiment 48. A kit comprising: the photoactivatable drug conjugate of any one of embodiments 1-37; and a light delivery unit of any one of embodiments 39-47.

Exemplary embodiment 49. A method of treating an aneurysm in an individual, the method comprising: (a) administering a photosensitizer; and (b) photoactivating the photosensitizer at the site of or near the aneurysm.

Exemplary embodiment 50. The method of embodiment 49, wherein the photosensitizer comprises the photosensitizer of any one of embodiments 1-37.

Exemplary embodiment 51. A method of treating an aneurysm in an individual, the method comprising: (a) administering the photoactivatable drug conjugate of any one of embodiments 1-37 to the individual; and (b) photoactivating the photosensitizer.

Exemplary embodiment 52. The method of embodiment 51, wherein (b) comprises photoactivating the photosensitizer at the site of or near the aneurysm.

Exemplary embodiment 53. The method of cany one of embodiments 51-52, wherein the photoactivatable drug conjugate localizes to regions near or within tissue inflammation or an aneurysm, as compared to a region of healthy tissue.

Exemplary embodiment 54. The method of any one of embodiments 49-53, wherein photoactivating the photosensitizer:
(i) reduces the rate of aneurysms growth and/or reduces/maintains the diameter a blood vessel having an aneurysm, as compared to an untreated blood vessel having an aneurysm;
(ii) decreases the volume of an aneurysm or reduces rate of increase in the volume of the aneurysm, as compared to an untreated aneurysm;
(iii) reduces the rate of blood vessel diameter growth in an blood vessel having an aneurysm, as compared to an untreated blood vessel having an aneurysm; and/or
(iv) decreases the rate of volume increase in an aneurysm or inhibits an increase in the volume of the aneurysm, as compared to an untreated aneurysm.

Exemplary embodiment 55. The method of embodiment 54, wherein the blood vessel is the aorta.

Exemplary embodiment 56. The method of any one of embodiments 49-55, wherein the aneurysm is an aortic aneurysm, a cerebral aneurysm, a thoracic aortic aneurysm, an abdominal aortic aneurysm, a popliteal artery aneurysm, a peripheral aneurysm, a fusiform aneurysm, a saccular aneurysm, a mycotic aneurysm, a pseudo aneurysm.

Exemplary embodiment 57. The method of any one of embodiments 49-56, wherein the aneurysm is an aortic aneurysm.

Exemplary embodiment 58. The method of any one of embodiments 49-57, wherein photoactivating in (b) is achieved by a catheter.

Exemplary embodiment 59. The method of embodiment 58, wherein the catheter is inserted through the groin artery.

Exemplary embodiment 60. The method of any one of embodiments 49-59, wherein the photosensitizer or photoactivatable drug conjugate is administered by parenteral injection.

Exemplary embodiment 61. The method of embodiment 60, wherein the photosensitizer or photoactivatable drug conjugate is administered by intravenous injection.

Exemplary embodiment 62. A method of treating ECM degradation in an individual, the method comprising: (a) administering to the photoactivatable drug conjugate of any one of embodiments 1-37; and (b) photoactivating the photosensitizer.

Exemplary embodiment 63. A method of treating tissue degeneration in an individual, the method comprising: (a) administering to the photoactivatable drug conjugate of any one of embodiments 1-37; and (b) photoactivating the photosensitizer.

Exemplary embodiment 64. A method of treating tissue degeneration in an individual, the method comprising: (a) administering a photosensitizer to an individual; and (b) photoactivating the photosensitizer at a region of the tissue degradation.

Exemplary embodiment 65. The method of any one of embodiments 62-48, wherein photoactivating the photosensitizer increases crosslinking of the ECM.

Exemplary embodiment 66. The method of any one of embodiments 62-49, wherein photoactivating the photosensitizer reduces and/or inhibits tissue degradation.

Exemplary embodiment 67. The method of any one of embodiments 62-51, wherein photoactivating the photosensitizer reduces and/or inhibits a loss of tissue thickness or degradation.

Exemplary embodiment 68. The method of any one of embodiments 62-52, wherein the

Exemplary embodiment 69. A method of treating a disease or disorder characterized by tissue degeneration in an individual, the method comprising: (a) administering to the photoactivatable drug conjugate of any one of embodiments 1-37; and (b) photoactivating the photosensitizer, thereby increasing tissue strength at the site of photoactivation.

Exemplary embodiment 70. The method of embodiment 69, wherein the tissue degeneration is characterized by weakened tissue and/or degraded extracellular matrix.

Exemplary embodiment 71. The method of embodiment 70, wherein the weakened tissue and/or degraded extracellular matrix is characterized by collagen and/or elastin degradation.

Exemplary embodiment 72. The method of any one of embodiments 69-71, wherein the tissue degeneration occurs at or within an organ, a joint, a blood vessel, a tendon, or a fascia.

Exemplary embodiment 73. The method of any one of embodiments 69-72, wherein the tissue degeneration occurs at or within a blood vessel.

Exemplary embodiment 74. The method of any one of embodiments 69-73, wherein the disease or disorder is a cardiovascular disease, an aneurysm, an inflammatory disease.

Exemplary embodiment 75. The method of embodiment 74, wherein the aneurysm is an aortic aneurysm, a cerebral aneurysm, a thoracic aortic aneurysm, an abdominal aortic aneurysm, a popliteal artery aneurysm, a peripheral aneurysm, a fusiform aneurysm, a saccular aneurysm, a mycotic aneurysm, a pseudo aneurysm.

Exemplary embodiment 76. The method of embodiment 74, wherein the aneurysm is an abdominal aortic aneurysm.

Exemplary embodiment 77. The method of any one of embodiments 69-76, wherein increasing tissue strength increases cross-linking of the ECM.

Exemplary embodiment 78. The method of any one of embodiments 69-77, wherein increasing tissue strength reduces immune cell infiltration into tissue.

Exemplary embodiment 79. The method of any one of embodiments 69-78, wherein increasing tissue strength is characterized by retention of collagen and/or elastin.

Exemplary embodiment 80. The method of any one of embodiments 69-79, wherein increasing tissue strength results in a decrease or cessation in a rate of artery diameter growth.

Exemplary embodiment 81. The method of any one of embodiment 69-80, wherein increasing tissue strength results in stabilizes artery diameter size.

Exemplary embodiment 82. The method of any one of embodiments 69-81, wherein increasing tissue strength results in reduced growth of the aneurysm.

Exemplary embodiment 83. The method of any one of embodiments 69-82, wherein increasing tissue strength results in a decrease or cessation in growth rate of the aneurysm.

Exemplary embodiment 84. The method of any one of embodiments 69-83, wherein increasing tissue strength stabilizes the aneurysm.

Exemplary embodiment 85. The method of any one of embodiments 69-84, wherein increasing tissue strength is characterized by retention the tunica adventitia, tunica intima, and/or tunica media.

Exemplary embodiment 86. The method of any one of embodiments 69-85, wherein the composition is administered intravenously or orally.

Exemplary embodiment 87. The method of any one of embodiments 69-86, wherein the light is administered by a laser catheter.

Exemplary embodiment 88. The method of any one of embodiments 69-87, wherein the light is administered at an irradiance of about 1 mW/cm² to about 600 mW/cm².

Exemplary embodiment 89. The method of any one of embodiments 69-88, wherein the light is administered at an irradiance of about 25 mW/cm² to about 100 mW/cm².

Exemplary embodiment 90. A photoactivatable drug conjugate comprising anakinra attached to methylene blue.

Exemplary embodiment 91. A photoactivatable drug conjugate comprising anakinra attached to indocyanine green.

Exemplary embodiment 92. A photoactivatable drug conjugate comprising anakinra attached a photosensitizer selected from: a porphyrin, a chlorin, a pheophorbide, a bacteriopheophorbide, a phthalocyanine, an anthraquinone, a phenothiazine, a xanthene, or a cyanine.

Exemplary embodiment 93. A method of increasing crosslinking within the extracellular matrix; the method comprising:
(a) administering a photosensitizer; and
(b) photoactivating the photosensitizer at the site of or near tissue degradation, ECM degradation, or an aneurysm.

Exemplary embodiment 94. The method of embodiment 93, wherein the photosensitizer comprises the photosensitizer of any one of embodiments 1-37.

Exemplary embodiment 95. A method of increasing crosslinking within the extracellular matrix; the method comprising:
(a) administering the photoactivatable drug conjugate of any one of embodiments 1-37 to the individual; and
(b) photoactivating the photosensitizer.

Exemplary embodiment 96. The method of embodiment 95, wherein (b) comprises photoactivating the photosensitizer at the site near or within tissue degradation, ECM degradation, or an aneurysm.

Exemplary embodiment 97. The method of any one of embodiments 95-96, wherein the photoactivatable drug conjugate localizes to regions near or within tissue degradation, ECM degradation, or an aneurysm.

Exemplary embodiment 98. The method any one of claims 93-97, wherein increasing ECM crosslinking is measured by reduced elastase-induced degradation in a treated tissue sample (as compared to an untreated sample) or an increase in-(carboxymethyl) lysine (CML) (as compared to an untreated sample).

Exemplary embodiment 99. A method of increasing the mechanical strength of a tissue; the method comprising:
(a) administering a photosensitizer; and
(b) photoactivating the photosensitizer at the site of or near tissue degradation, ECM degradation, or an aneurysm.

Exemplary embodiment 100. The method of embodiment 99, wherein the photosensitizer comprises the photosensitizer of any one of embodiments 1-37.

Exemplary embodiment 101. A method of increasing the mechanical strength of a tissue; the method comprising:
(a) administering the photoactivatable drug conjugate of any one of embodiments 1-37 to the individual; and
(b) photoactivating the photosensitizer.

Exemplary embodiment 102. The method of embodiment 101, wherein (b) comprises photoactivating the photosensitizer at the site near or within tissue degradation, ECM degradation, or an aneurysm.

Exemplary embodiment 103. The method any one of claims 99-102, wherein increasing the mechanical strength of the tissue is measured by the mechanical strength (e.g., forceps test) and/or stiffness of a porcine aorta subjected to elastase.

Exemplary embodiment 104. The method any one of claims 99-103, wherein the tissue is an inflamed tissue or degraded tissue or within or near the site of an aneurysm.

Exemplary embodiment 105. A method for treating an aneurysm comprising: administering to a subject in need thereof an activatable drug conjugate wherein the activatable drug conjugate comprises a targeting moiety for targeting the conjugate to an inflamed tissue and an activatable agent that is activated via an energy source; and administering energy from the energy source to a site at or near the aneurysm.

### EXAMPLES

### Example 1: Methylene Blue Photodynamic Therapy Using Methylene Blue Inhibits the Induction of AAA

To assess if methylene blue (MB) photodynamic therapy (PDT) can exert a protective benefit against the development of abdominal aortic aneurysms (AAAs), an aneurysm was induced via surgery in a BAPN-Elastase Mouse AAA Model. Briefly, mice were maintained on standard diet and given 0.2% β-Aminopropionitrile (BAPN) water, as previously described. Aneurysms were induced through a surgical procedure, where the infrarenal aorta was isolated and treated with porcine pancreatic elastase (PPE), then rinsed, to induce aneurysms. Immediately following aneurysm induction, the site of the aneurysm was treated with MB (unconjugated) PDT by direct topical administration of MB to the aneurysm and applying light. MB has a structure represented by the formula:

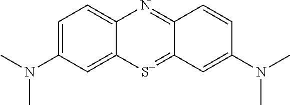

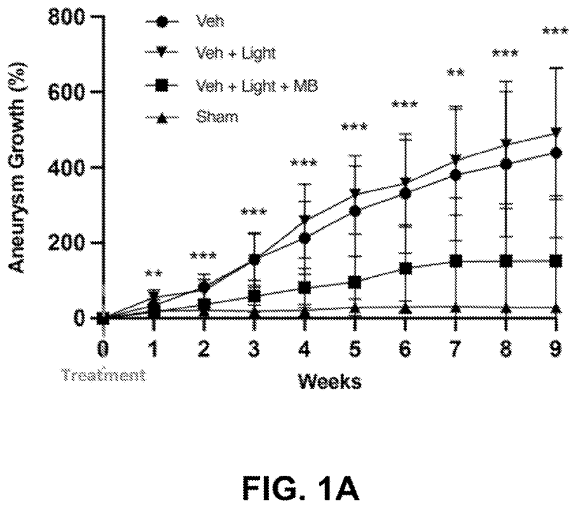

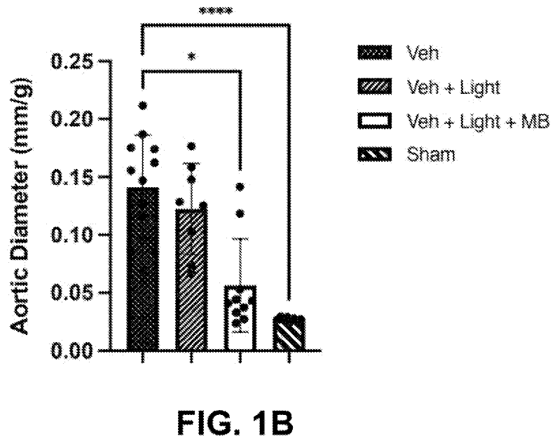

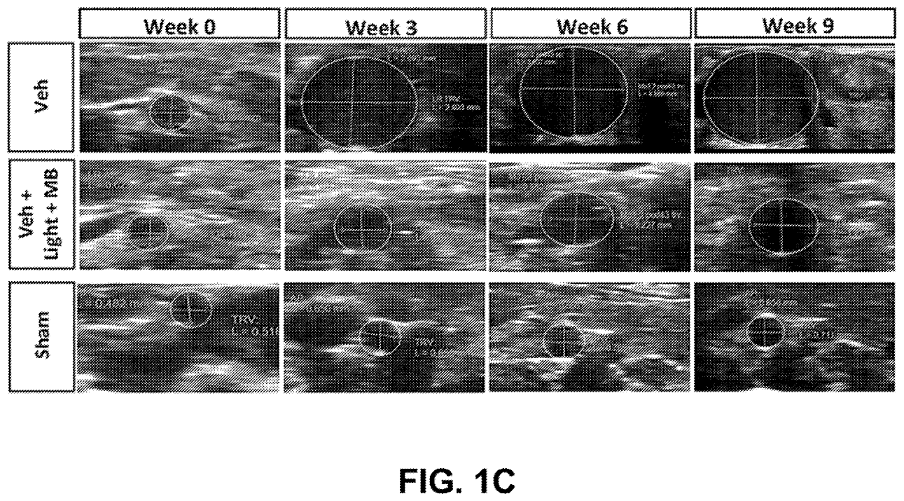

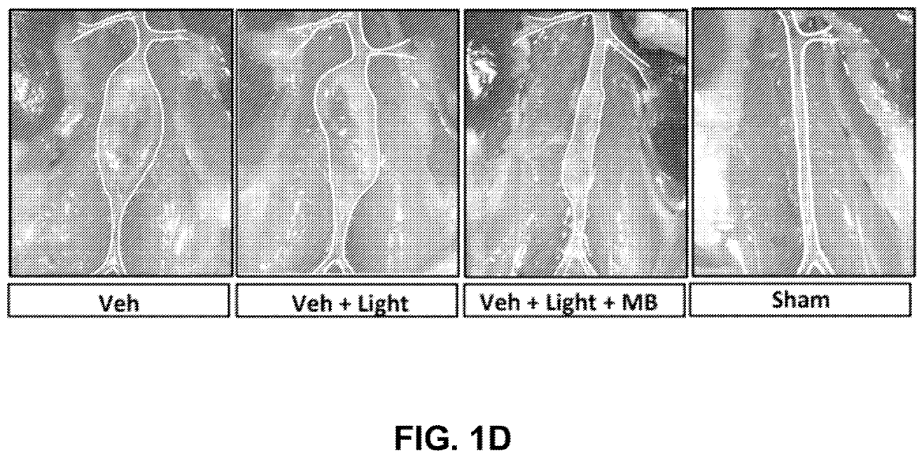

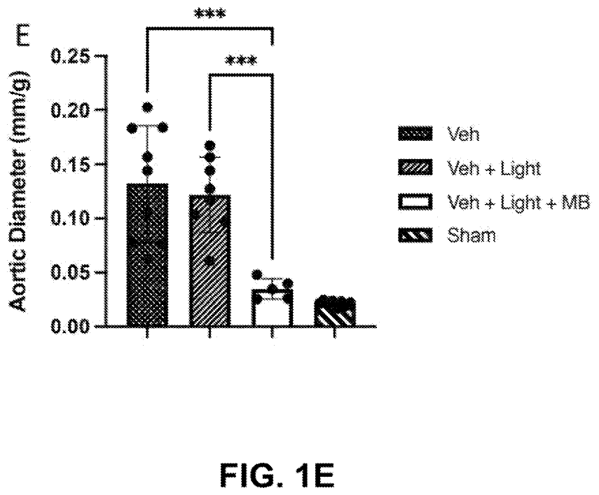

Vehicle group were mice topically treated with elastase and then Hank's balanced salt solution (HBSS) without light and considered as AAA positive control. Mice topically treated with HBS and no light were utilized as light only controls. Sham group were mice that underwent the same AAA surgery but were treated with heat-inactivated PPE are considered as healthy controls. Following treatment, mice were monitored for aneurysm growth through weekly ultrasound surveillance over a period of 9 weeks post-induction surgery (FIG. 1A). For ultrasound surveillance, maximum aortic diameters were measured by averaging anterior-posterior (AP) and transverse (TRV) dimensions. MB PDT treatment significantly restricted AAA growth (FIG. 1A). In stark contrast to the aneurysms growth observed in vehicle-treated group, the average increase in size of the PDT-treated group (Veh+light+MB) was significantly lower. (FIG. 1B) Similar to vehicle only, no benefit was observed in the light only group. This suggests that MB PDT not only limits AAA formation but also that this protective benefit extends over the duration of the study post-treatment. Morphological images and micrometry quantifications of treated and untreated AAAs at the end of the study corroborated the ultrasound findings, with marked differences apparent between the two groups (Veh versus Veh+light+MB) (FIGS. 1C and 1D). These results collectively show that MB PDT (a method of treating an aneurysm in an individual, the method comprising: (a) administering a photosensitizer; and (b) photoactivating the photosensitizer using light at the site of or near the aneurysm) serves as an effective intervention to mitigate AAA development.

### Methods—BAPN-Elastase Mouse Model:

The animal protocols were approved by the University of California San Francisco Institutional Animal Care and Use Committee. AAA Induction protocol was taken from established methods and was chosen for its ability to consistently induce aneurysms (100% of mice) and mimic similar inflammatory pathology to human aneurysms (1,2,3). 6-8 week male C57BL/6J wild type mice (n=x) were kept on standard chow diet throughout the course of the experiment. Starting from two days before surgery until the end of the experiment, mice were kept on 0.2% B-Aminopropionitrile (BAPN; A3134-25G) drinking water. BAPN is a lysyl oxidase inhibitor that reduces collagen crosslinking in tissue. At the time of surgery, a laparotomy is performed to expose the infrarenal aorta. The infrarenal aorta is then carefully separated from the IVC by blunt dissection. 5 ul of Porcine Pancreatic Elastase (PPE; E1250-25 MG) is applied topically onto the aorta using a pipette. After 5 min, the PPE was washed away using two changes of warmed sterile saline (RGC-3290). After the wash, further treatment can be done as described in the following examples. Then, the abdominal incision is closed and animals allowed to recover. Unless otherwise specified, the methods described in this section can be similarly applied to similar experiments throughout the examples.

### Methods—MB-PDT Treatment:

The photosensitizer dye methylene blue (M291-100) was used for photodynamic therapy treatment (MB-PDT). MB-PDT was applied at two time points during the experiment. To test the effect of MB-PDT on AAA formation, MB-PDT was done directly after the PPE saline wash (PODO). To investigate the effect of MB-PDT on AAA growth, Laparotomy and MB-PDT was performed 21 days postoperatively, covering the whole aneurysm (POD21). MB-PDT involves applying 10 ul of 10:1 MB Solution in DPBS (M291-100:21-030-CV) topically to the whole aneurysm, which was carefully dissected from surrounding tissue. Gauze was placed on either side of the aneurysm to absorb excess MB. The aneurysm was then irradiated for 25 min at an irradiance of 30 mW/cm\hat{ }2 and fluence of 45 J/cm\hat{ }2 (660 nm LED, Thor M660L4). Throughout the course of irradiation, 5 ul of MB was applied to the aneurysm every 2.5 min to prevent the tissue from drying out. After irradiation, the MB was washed with two changes of sterile saline. For the MB only group, the total amount of MB was applied topically to the aneurysm for five minutes and washed. For the light only group, the aneurysm was irradiated for 25 min, with 5 ul of sterile saline applied every 2.5 min to prevent tissue desiccation. For POD21 positive controls, a laparotomy was performed with no additional treatment. Unless otherwise specified, the methods described in this section can be similarly applied to similar experiments throughout the examples.

### Methods—Ultrasound Surveillance:

Aneurysm size was tracked by weekly ultrasound surveillance. Animals were anesthetized and placed on a heated pad during the procedure (2-4% isoflurane, 1 L/min medical grade air). A Vevo 3100 system with a 32- to 55-MHz frequency transducer was used to collect standard B-mode images in long axes (Fujifilm VisualSonics, Toronto, ON, Canada; MS550D, 40-MHz center frequency). After obtaining the picture, the anterior to posterior diameter and the transverse diameter were both measured and recorded. Weekly growth of aneurysm size was calculated by subtracting the previous week's measurement of maximum aortic diameter (in mm) from the current week. The first three week's calculation was averaged to represent the average weekly growth rate of aneurysms before treatment. The next six weeks (POD 21-63) were averaged to represent the average weekly growth rate of aneurysms after treatment. Unless otherwise specified, the methods described in this section can be similarly applied to similar experiments throughout the examples.

### Methods—Micrometry:

During the harvest procedure on POD63, mice were placed under anesthesia and opened via laparotomy. Three in vivo micrometry measurements was taken of the pressurized aneurysm at maximum diameter using the Leica X Microscope system and averaged. The averaged measurement was then normalized to total body weight of the mouse at time of harvest. Unless otherwise specified, the methods described in this section can be similarly applied to similar experiments throughout the examples.

### Methods—Statistics

Statistics tests were performed in GraphPad Prism version 7.0 (GraphPad Software). Unless indicated otherwise, values are presented as mean±SD. All data were tested for normality and equal variance. If the data passed those tests, Student t test was used to compare 2 groups. One-way ANOVA followed by Kruskal-Wallis test post hoc analysis was used for comparisons among >2 groups. If the data did not pass those tests, the Mann-Whitney U test was used to compare 2 groups, and the Kruskal-Wallis test for >2 groups. P<0.05 was considered statistically significant. Unless otherwise specified, the methods described in this section can be similarly applied to similar experiments throughout the examples.

### Example 2: MB PDT Modulates Immune Cell Infiltration and Apoptosis

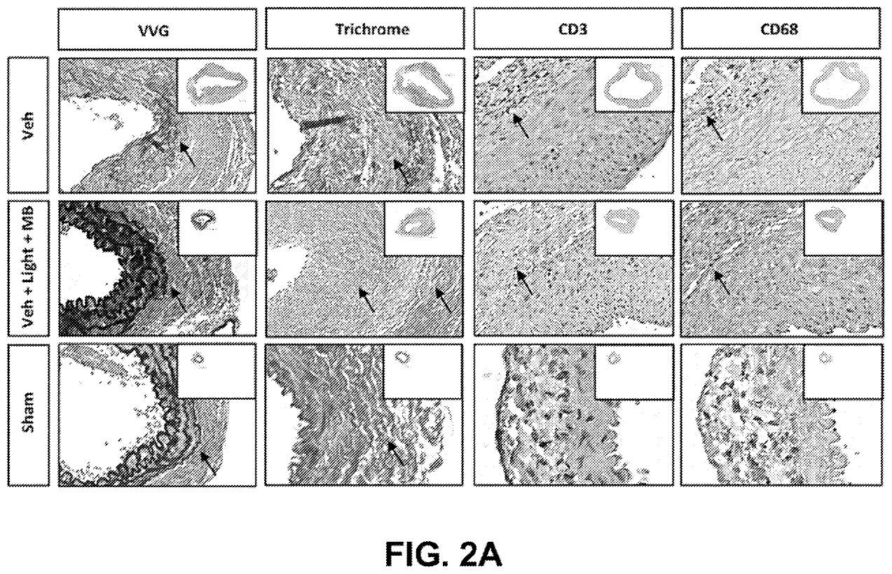

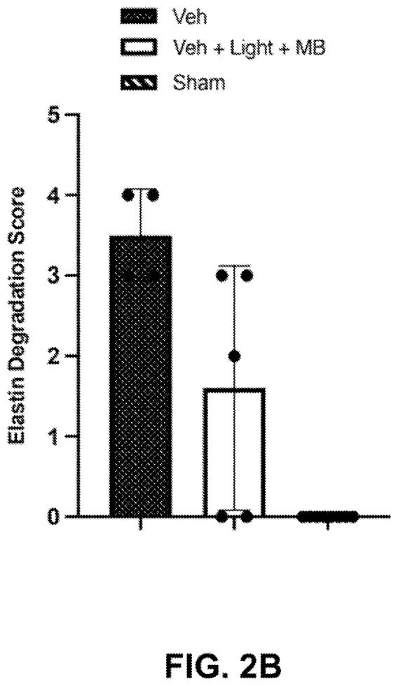

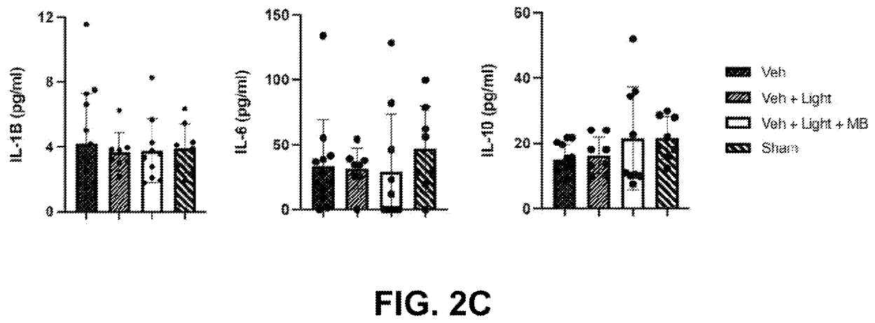

The formation and progression of AAA can be characterized by the degradation of the extracellular matrix (ECM) and the concurrent infiltration of immune cells into the aortic wall. To assess if MB PDT affects the extracellular matrix (ECM) of the aortic aneurysms, cross-sections of the aneurysms of the above mice which have undergone AAA induction and concurrently treated with MB PDT and the respective controls were stained with Verhoeff-Van Gieson (VVG) for elastin, Massons Trichrome for collagen and CD3 antibody for T-cells and CD68 antibody for macrophages for immune cell infiltration (FIG. 2A). Upon magnification, less elastin staining was observed (pointed by arrows) in the lumen after MB PDT treatment compared to vehicle treated controls indicative of a reduction of elastin degradation after PDT. Interestingly the staining for elastin after MB PDT was stronger (pointed by arrows) than healthy sham controls suggesting an increase in elastin production as a compensatory mechanism. Similarly, a compensatory effect of collagen expression into the elastin layer was observed (pointed by arrows) after PDT treatment compared to both vehicle-treated positive controls and sham controls. In line with this, less collagen degradation was also observed in the adventitia after PDT treatment compared to vehicle treated controls and sham mice. Mice treated with vehicle only had an overall decrease in collagen staining in the adventitia suggesting a breakdown of adventitial collagen. To assess if T-cells and macrophage infiltration is modulated in aneurysms upon treatment with MB PDT, the aneurysm cross-sections were stained for CD3 and CD68, markers for T-cells and macrophages, respectively. CD3 and CD68 staining were reduced after MB PDT treatment compared to vehicle treated controls (FIG. 2A). Little to no staining was observed in sham surgery mice suggesting that immune cell infiltration is increased in aneurysm formation and treatment with MB PDT inhibits immune cell infiltration. To confirm if there is an improvement in elastin degradation, clinical scoring as per an established protocol was performed by blinded investigators. MB PDT treated group showed a trend towards improvement against elastin degradation (FIG. 2B). To assess if this benefit on elastin degradation and immune cell infiltration was due to any systemic decrease in circulating cytokine levels known to be associated with aneurysm growth, cytokines such as IL-1B, IL-6 and IL-10, serum cytokine levels were measured. No significant differences were observed in serum cytokine levels suggesting that the improvement is due to a localized effect of MB PDT treatment on aneurysmal tissue inflammation (FIG. 2C).

These results collectively show that MB PDT (a method comprising: (a) administering a photosensitizer; and (b) photoactivating the photosensitizer using light) serves as an effective intervention to decease elastin degradation and reduce immune cell infiltration.

### Methods—Histology and IHC Staining:

Fresh tissue was fixed in 10% neutral buffered formalin (Azer Scientific, PFNBF-90) for 24 hours before being transferred to 70% EtOH. Storage in 70% EtOH did not exceed one month. After paraffin embedding, sections were cut at 5 um for staining (AML Laboratories). Sections were then stained with H&E (VWR 95057-844, 95057-848), Van Gieson (Statlab Medical Products, KTVELLT), and Masson's Trichrome (CP Lab Chemicals, TRM-500). For IHC, standard immunohistochemistry procedures for 5 μm-thick aorta sections were used. In brief, after deparaffinization, endogenous peroxidase activity was quenched by placing the slides into 3% hydrogen peroxide (H2O2) for 10 min. Sections were immersed in preheated 10 mM citric acid (VWR., Radnor, PA., USA), pH 6.0, for 30 min and cooled in cold water. Sections were then blocked in 5% goat serum (Vector Laboratories, Burlingame, Calif., USA) for 20 min before being incubated overnight at 4° C. in a solution of rabbit anti-CD3 (0.4 μg/ml; ab5690; Abcam, Cambridge, MA., USA) antibody, rabbit anti-CD31 (ERP1729; 0.4 μg/ml; ab182981; Abcam) antibody, rabbit alpha smooth muscle Actin (SMA; 0.067 μg/ml; ab5694; Abcam), rabbit anti-CD68 (ERP23917; 2.0 μg/ml; ab283667; Abcam), and rabbit anti-Interleukin-1 receptor type 1 (IL1R1; 2.0 μg/ml; PAS-98766; Thermo Fisher, Eugene, OR., USA) in PBS. The next day, the sections were exposed to the biotinylated biotinylated goat-anti-rabbit IgG secondary antibody (15 μg/ml; Vector Laboratories) in PBS for 1 h followed by avidin-biotin complex for 1 h (1:200, ABC; Vector Laboratories). The reactions were visualized with 3,3′-diaminobenzidine (Millipore-Sigma) for 10 min and counterstained with Gill's Haematoxylin (Thermo Scientific) for 30 seconds. Finally, the sections were dehydrated, cleared in xylene and cover slipped. As negative controls, staining was performed in the absence of the primary antibodies and no specific staining was identified in these preparations. In addition, a sample of mouse skin and mouse lung was used as positive controls and compared the immunoreactivity.

For quantification of elastin using Van Gieson, an elastin scoring guide was used. A score of 0 was assigned to samples with no elastin breakage, I was given to elastin break/degradation or SMC loss limited to one outer medial elastin layer, II for elastin degradation or SMC loss involving more than two medial elastin layers, or entire medial elastin layers, but limited to less than one-quarter of the aortic circumference, III for elastin degradation or SMC loss involving entire medial elastin layers, but limited to less than half the aortic circumference, and IV for elastin degradation or SMC loss involving entire medial elastin layers and expanded to more than three-quarters of aortic circumference. Unless otherwise specified, the methods described in this section can be similarly applied to similar experiments throughout the examples.

### Methods—ELISAs:

Complete whole blood was collected from each mouse via puncture of the left ventricle of the heart. After 5 mins, the blood was centrifuged and serum supernatant was collected and stored at −80 C. Tissue samples were lysed by a French press. ELISAs for Anakinra (IL-1 receptor antagonist) IL1 receptor, IL-10, IL1B, IL6, elastin and CML were run according to the manufacturer's instructions. Absorbance was read on a microplate reader at recommended wavelengths (Molecular Devices SpectraMax M2). Unless otherwise specified, the methods described in this section can be similarly applied to similar experiments throughout the examples.

Methods—Ex Vivo Elastase Degradation Assay with VG Staining:

Human and porcine aorta were thawed from −80 C to RT. Tissue was then cut into ~1 cm×1 cm squares and briefly rinsed with HBSS. Tissues were then split into two groups: control and PDT. Control tissues were kept in HBSS at RT for one hour, and treated tissues were subjected to 150 mW NIR light for 20 mins in 100 μg/ml methylene blue, with the lumen side facing the light. Then each group was further split into two groups, no elastase and elastase treatment. Treatment involved a 2 hour incubation at 37 C in 10% Porcine Pancreatic Elastase (E1250-25 MG). After treatment all tissues were briefly rinsed, dried on a paper towel, and frozen in OCT. Tissue was sectioned at 10 μm by cryotome for the cross-section and stained using Van Gieson (Statlab KTVELLT). Images were taken at 20× and 100× on a Keyence BZ-X700 Fluorescence Microscope. Unless otherwise specified, the methods described in this section can be similarly applied to similar experiments throughout the examples.

### Example 3: MB PDT Extends Therapeutic Window for Reducing the Growth of AAA

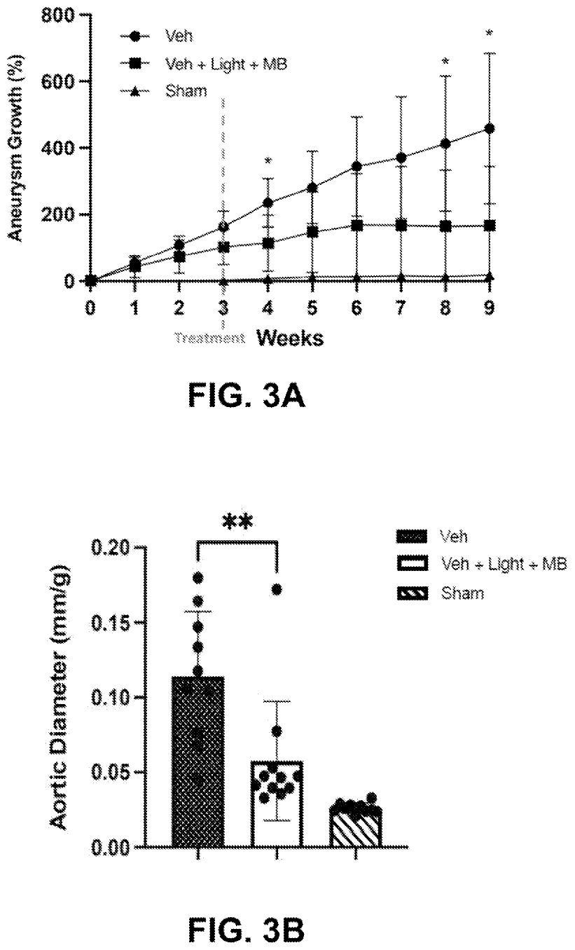

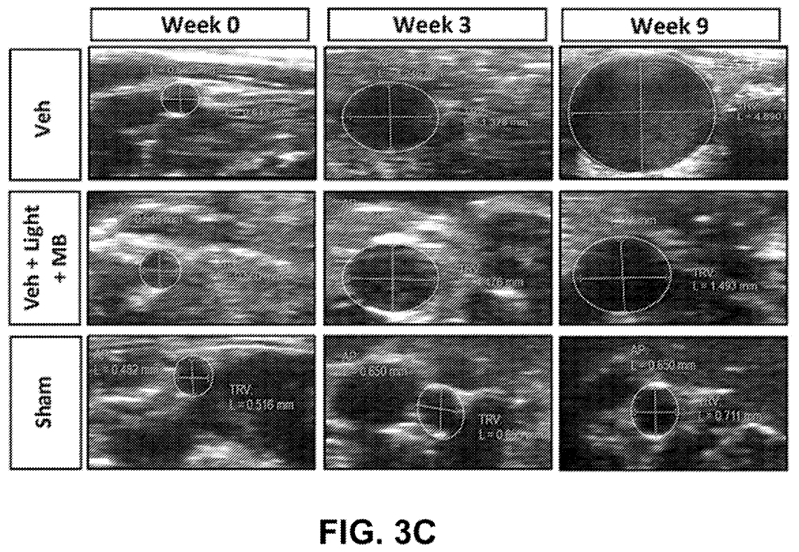

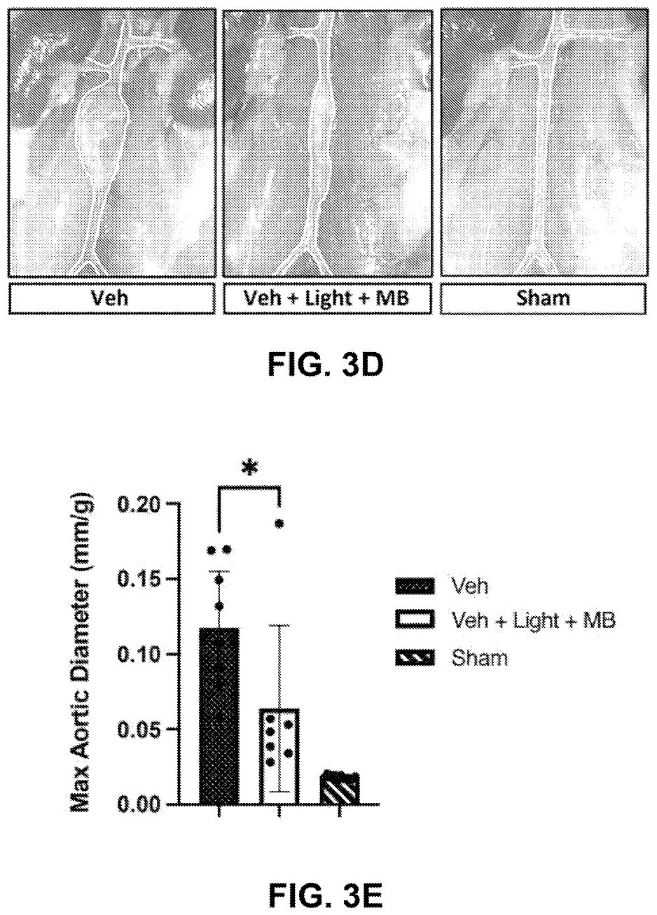

In certain instances, AAA often goes undiagnosed or is diagnosed late, thus it is important to know if MB PDT can not only prevent AAA formation but also reduce or arrest progressive aneurysm growth. To evaluate the efficacy of MB PDT to inhibit the progression of AAA, MB PDT was administered to mice with pre-established and progressive aneurysms 21 days after the initial aneurysm induction surgery with elastase. Vehicle group were mice which have undergone AAA induction surgery and topically treated with Hank's balanced salt solution (HBSS) without light and considered as AAA positive controls. Sham group were mice that underwent the same AAA surgery but were treated with heat inactivated PPE and considered healthy controls. Analysis of growth curves based on weekly ultrasound measurements revealed that MB PDT significantly reduced the enlargement of established AAAs in mice (FIG. 3A) compared to vehicle-treated controls. The aneurysm enlargement after week 3 for those animals treated with MB PDT was significantly less than that observed in the vehicle-treated group. Similar to the measurements done in the study of MB PDT upon AAA induction in FIG. 1, the maximum aortic diameters were measured using ultrasound, averaging anterior-posterior (AP) and transverse (TRV) dimensions for accuracy. The decrease in size of aneurysm upon MB PDT treatment was corroborated by supporting findings from the ultrasound-derived data (FIG. 3 B,C). The healthy sham group, vehicle-treated group and MB PDT-treated groups had similar aortic diameter on day of AAA-induction surgery. A equivalent increase in aneurysm diameter was observed on week 3 between vehicle and MB PDT groups prior to treatment, indicating similar rate of AAA growth pre-treatment. However, upon MB PDT treatment, a decrease in aneurysm diameter was observed on week 9 compared to vehicle treated group (FIG. 3C), suggesting that PDT-treatment prevents an increase in aortic aneurysm size. No changes in aortic size were observed in healthy sham controls. Similar to mice treated immediately upon induction of AAA in mice, morphological images and the micrometric measurements of treated and vehicle AAAs corroborated the ultrasound findings, with marked differences apparent between the two groups (FIGS. 3C and 3D), suggesting that, MB PDT slows the rate of AAA growth in mice. These results collectively show that MB PDT effectively reduces the growth of the aneurysm.

### Example 4: Synthesis and Characterization of Anakinra Conjugated to Methylene Blue (Ana-MB)

To develop a modality for targeted delivery of methylene blue to the AAA site, methylene blue was conjugated to IL-1 receptor antagonist, Anakinra using an NHS or maleimide linker. Methods of linking a photoactivatable agent (e.g., methylene blue) to a protein (e.g., anakinra) are known in the art and can be used to form the photoactivatable drug conjugates described herein. To prepare Anakinra-methylene blue conjugates (Ana-MB) described in the examples, methylene blue NHS ester dye (3-(N, N-dimethylamino)-7-[N-(3-(N-succinimidyl)-carboxyethyl)-N-(methyl)-amino]phenothiazin-5-ium perchlorate) was dissolved in anhydrous DMSO to a final concentration of 10 mM. An anakinra (comprising SEQ ID NO: 1) solution of approximately 3.0 mg/mL was prepared in sodium borate buffer (50 mM, pH 8.5). For the conjugation step, while the dye solution was added to the protein solution at the appropriate molar ratio. The reaction was quenched with glycine (pH 7.4) after 4h. Following the reaction, any excess dye was removed using a desalting spin column.

To reveal the conjugation site, LC-MS/MS was performed on Ana-MB for peptide mapping. Lysine residues at positions 94 and 97 (denoted by * in the sequence) exhibited a tendency to undergo modification in tandem (FIG. 4A). The prevalence of peptides labeled at both sites was disproportionately high relative to the occurrence of peptides labeled at a single site (FIG. 4B). This observation aligns with the drug-loading profile observed in the intact mass analysis, which suggests a pattern of concurrent labeling. Within the primary structure of anakinra, the N terminal amine and lysine residues K7, K10, K46, K65, K94, and K97 (each further denoted by * in the sequence), were all subject to partial modification with methylene blue. Notably, none of these lysine sites are occluded within the binding interface between anakinra and the IL1 receptor (IL1R), indicating their accessibility for modification without impeding function.

### Example 5: Targeted Delivery of Methylene Blue Photosensitizer to Inflammatory Aneurysmal Tissue Improves Efficacy of Photodynamic Therapy

### Effective Targeting of Ana-MB

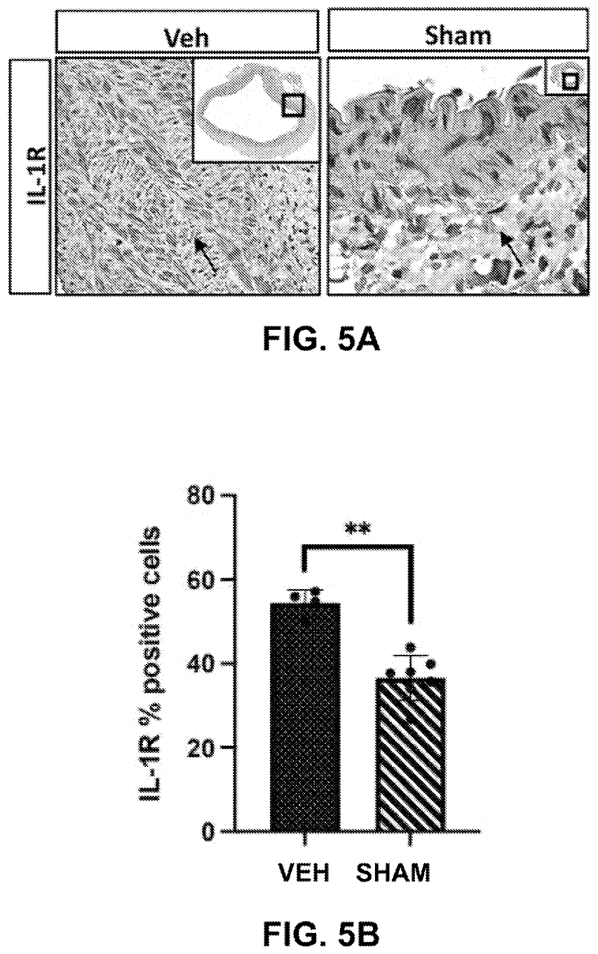

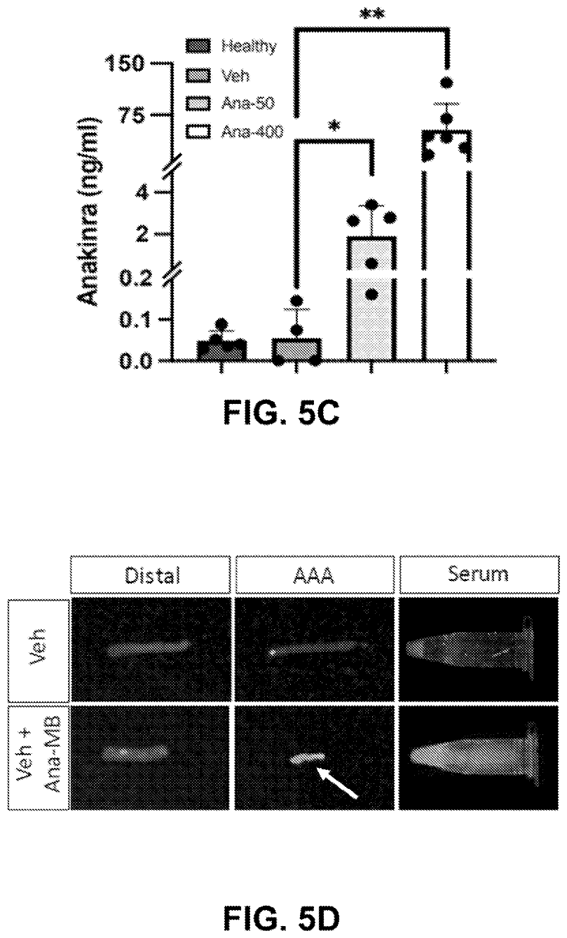

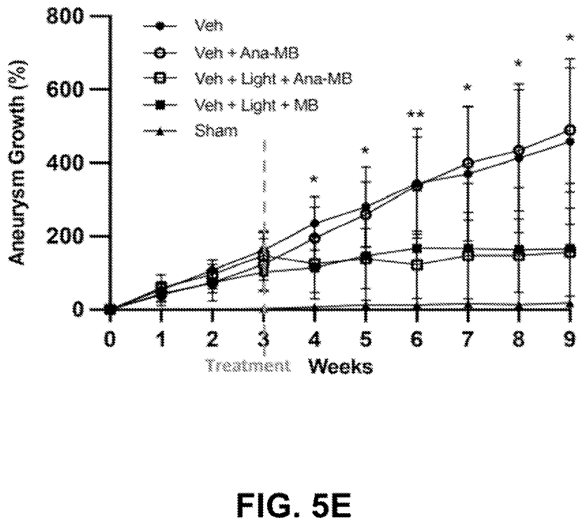

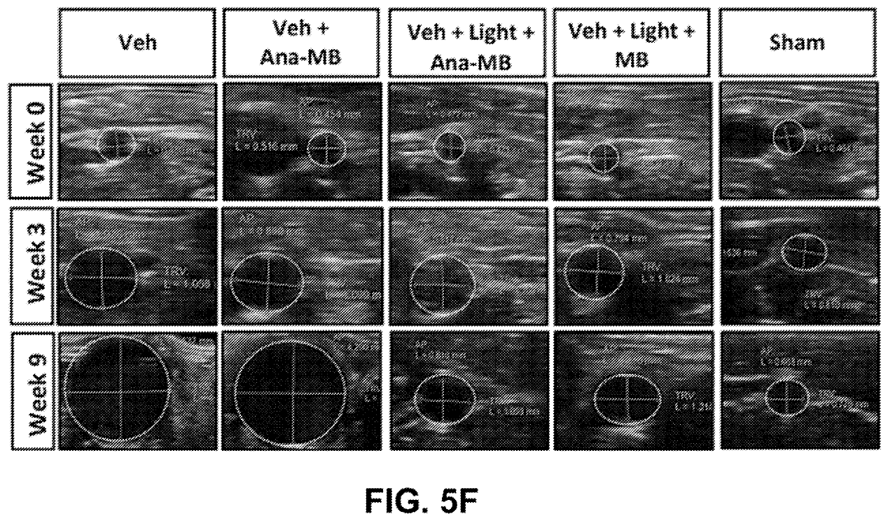

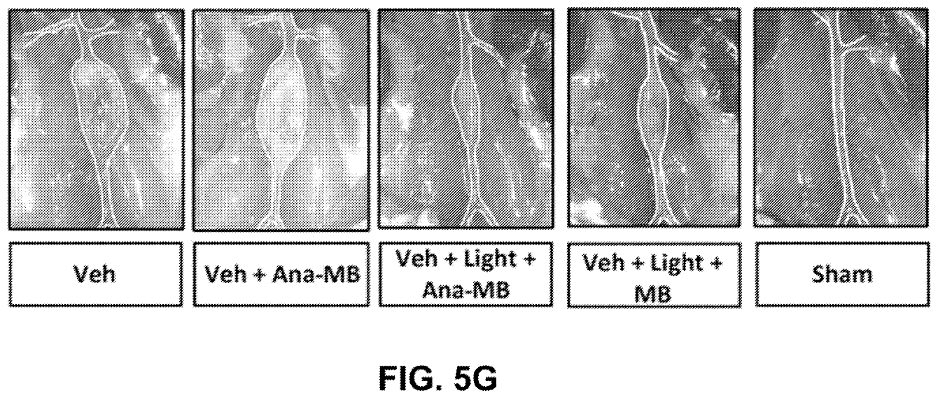

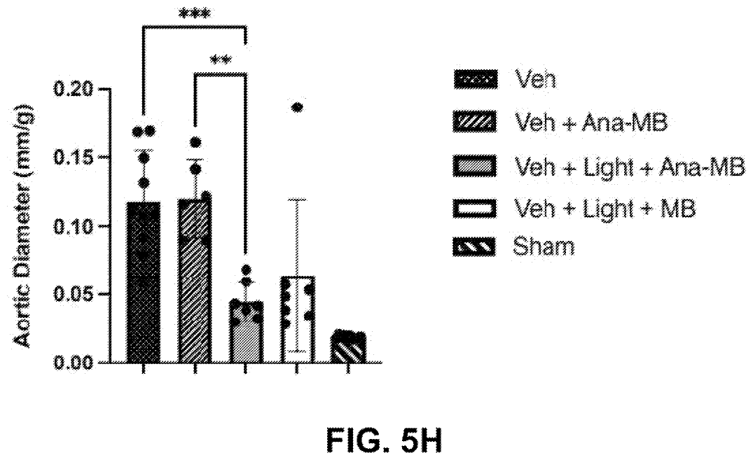

To ensure selective accumulation of methylene blue in aneurysmal tissue and by extension increase the potential therapeutic effect of PDT, a targetable activatable drug conjugate (ADC) comprising IL1R antagonist, Anakinra, conjugated to methylene blue (Ana-MB) was developed. This ensured the targeted delivery and localization of methylene blue at inflammatory sites within or near the site of aneurysmal tissue where IL-1 receptor is expressed, thus facilitating targeted PDT. To assess if IL1R is expressed in aneurysmal tissue, immunochemistry of IL1R was performed in healthy aortic tissue and aortic aneurysm cross-sections. Mice having undergone AAA surgery with active PPE were considered as vehicle and those treated with heat inactivated PPE were considered the healthy Sham control group. Staining for IL1R revealed significant increase in expression density of IL1R in aneurysms tissue compared to sham controls (FIGS. 5A and 5B). To assess if Ana-MB localizes to aortic aneurysmal tissue, mice with AAA and healthy sham controls with no AAA were treated with different doses of Ana-MB. Minimal anakinra accumulation was observed in aortic tissue of healthy mice treated with 50 μg anakinra, while a significant dose-dependent increase in anakinra accumulation was observed in mice with AAA, suggesting enhanced accumulation of Ana-MB in aneurysmal tissue (FIG. 5C). Similarly, fluorescence imaging was performed to visualize the localization of Ana-MB within aneurysm tissue compared to adjacent distal healthy aortic tissue. An increase in fluorescence caused by Ana-MB was observed in aneurysm tissue compared to adjacent healthy aortic tissue (FIG. 5D). In the vehicle treated group, no fluorescence was observed. Similarly increased fluorescence was observed from circulating Ana-MB present in serum compared to vehicle treated control, suggesting that while significant levels of drug is bound to the aneurysm site, there remains an abundant circulating drug reservoir poised for receptor binding within aneurysms following intravenous delivery. Unless otherwise specified, the methods described in this section can be similarly applied to similar experiments throughout the examples.

### Efficacy of Ana-MB PDT

At 21 days post-AAA-induction surgery, mice were treated with vehicle or topical MB applied on aortic wall (PDT group), or intravenously delivered Ana-MB without PDT, or intravenously delivered Ana-MB with PDT. The sham treated group was used as surgical controls. Treatment with Ana-MB+PDT completely inhibited the growth of AAA compared to Ana-MB without PDT treatment and vehicle treated positive controls. (FIG. 5E). The inhibition of AAA progression upon PDT treatment with intravenously delivered Ana-MB (Veh+Light+MB group) was comparable to topically applied MB group (Veh+Light+MB group) as shown in Examples 1, 3 (FIG. 1A, FIG. 3A). Furthermore, the quantified video microscopic measurement of AAA diameter supported the quantified ultrasound-derived conclusions from the duration of the study (FIG. 5F). Morphological and micrometric measurement comparisons (FIG. 5G, FIG. 5H) between AAAs treated with Ana-MB and photodynamic therapy 21 days post-induction and those receiving vehicle only treatment revealed a significant difference, affirming the efficacy of Ana-MB PDT in safeguarding against and decelerating AAA progression in murine models. Unless otherwise specified, the methods described in this section can be similarly applied to similar experiments throughout the examples.

### Methods—Localization of Anakinra Study:

Mice with aneurysms were injected with the following conditions on POD21: saline, Anakinra-50 μg, Anakinra-400 μg, and Anakinra-800 μg (n=4). 5 mice with healthy aorta was taken as an additional control. Aneurysms were harvested 30 minutes post injection and tissue was immediately flash frozen in liquid nitrogen and stored at −80° C. Serum was collected via cardiac puncture and stored at −80° C. For homogenization, tissue was placed in an Eppendorf with 2 mm beads and sample diluent buffer and lysed at 30 shakes/see for 10 minutes on the Tissue Lyser II. The eppendorfs were then centrifuged at max speed for 10 mins and supernatant was collected. Both serum and tissue lysate were used in the Human IL-1Ra ELISA and performed according to manufacturer's protocol (Ab211650). For fluorescence imaging mice were treated with 400 μg of Anakinra intravenously. Aortic aneurysm tissue, aortic tissue and serum were collected 30 min post-injection. Fluorescence imaging was performed using a Licor imaging device. Unless otherwise specified, the methods described in this section can be similarly applied to similar experiments throughout the examples.

### Example 6: MB PDT Protects Extracellular Matrix from Elastase-Induced Degradation Across Different Species

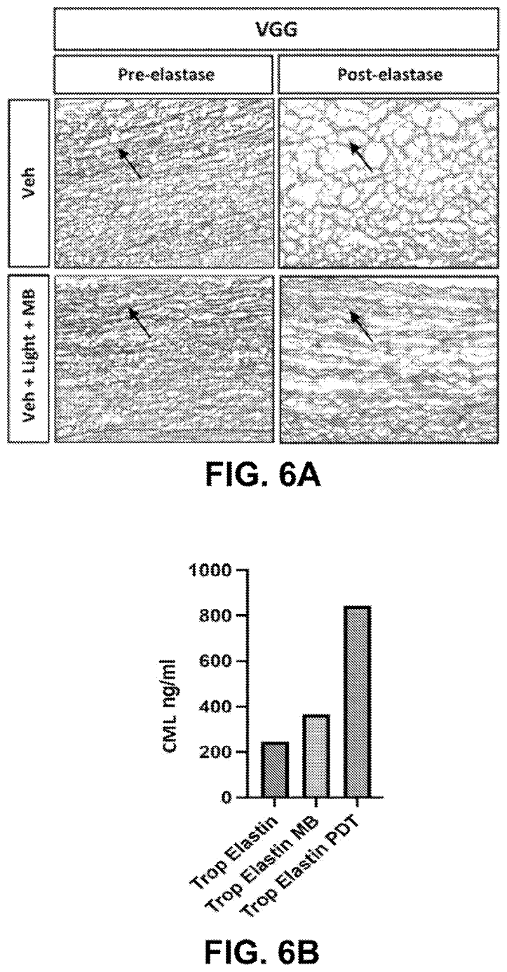

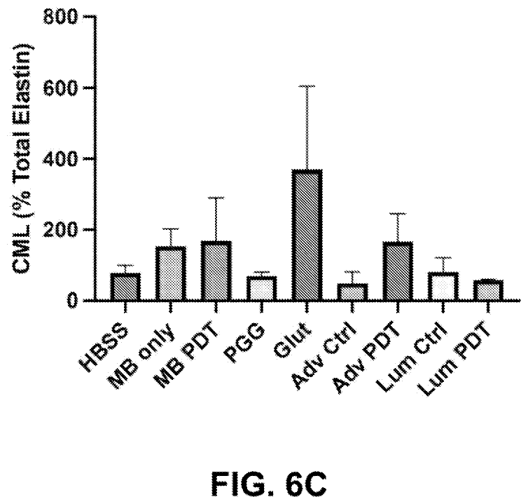

To assess if MB PDT can protect aortic tissue from elastase-induce extracellular matrix degradation, aortic tissue were procured from pigs and subjected to MB PDT (20 min irradiance of 150 mW/cm²) or Veh only treatment ex vivo before recombinant elastase treatment. Comparative analysis of the cross-sections of these tissues were sought to ascertain the consistency of PDT's protective effects across different biological species. Post-treatment analysis entailed staining of elastin with Verhoeff-Van Gieson (VVG) to evaluate resistance to elastin degradation in the aortic sections. Tissue treated with MB PDT showed significant resistance to elastase-induced ECM degradation as observed by an increase in the presence of elastin staining within the tissue and a decrease in tissue degradation as observed by a decrease in vacuoles and elastin breakage, suggesting that MB PDT-induced elastin crosslinking protects against elastase activity (FIG. 6A). Thus, the protective benefit of MB PDT on elastase induced-aortic degradation seems to be species independent. Ne-(carboxymethyl) lysine (CML), an elastin crosslinking modification, is a major modification caused by advanced glycation end (AGE) products in diabetes. Recombinant Tropoelastin treated with MB PDT showed a significant increase CML levels compared to untreated tropoelastin controls and tropoelastin incubated with methylene and untreated with light (FIG. 6B). Similarly, total elastin in porcine tissue showed a significant increase in CML modification levels after treatment with MB PDT compared to untreated samples and the positive control pentagalloyl glucose, which is a known potent chemical crosslinker, but less than glutaraldehyde another known chemical crosslinker (FIG. 6C). These data suggests that MB PDT induces elastin crosslinking within tissues.

### Example 7: MB PDT Improves Mechanical Strength of Aortic Tissue

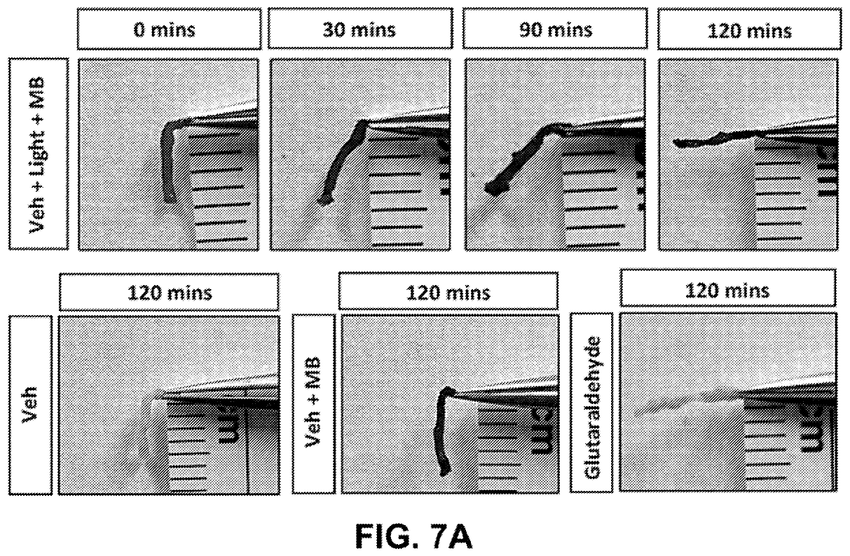

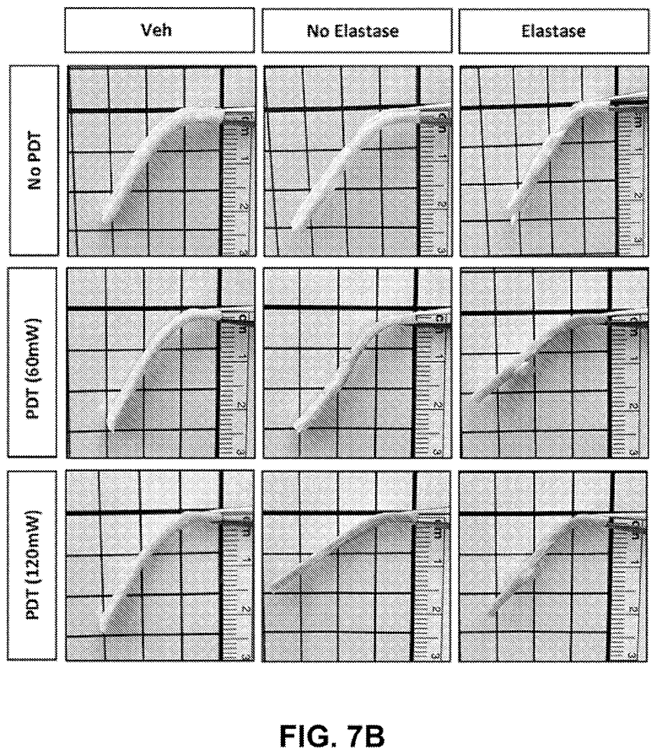

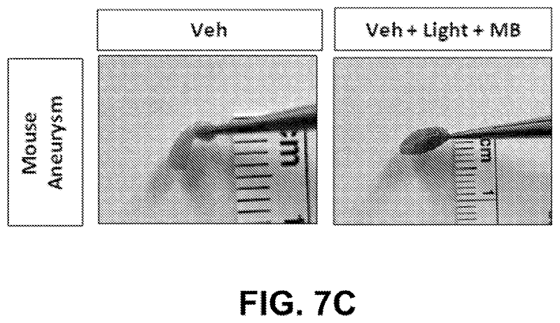

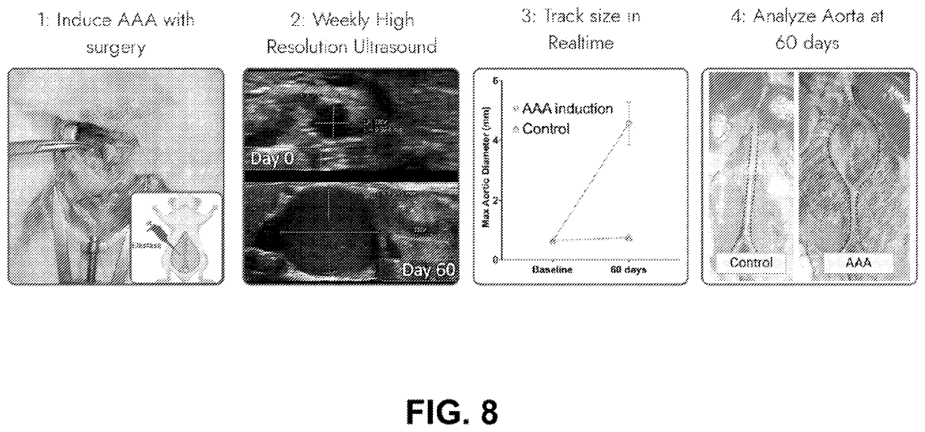

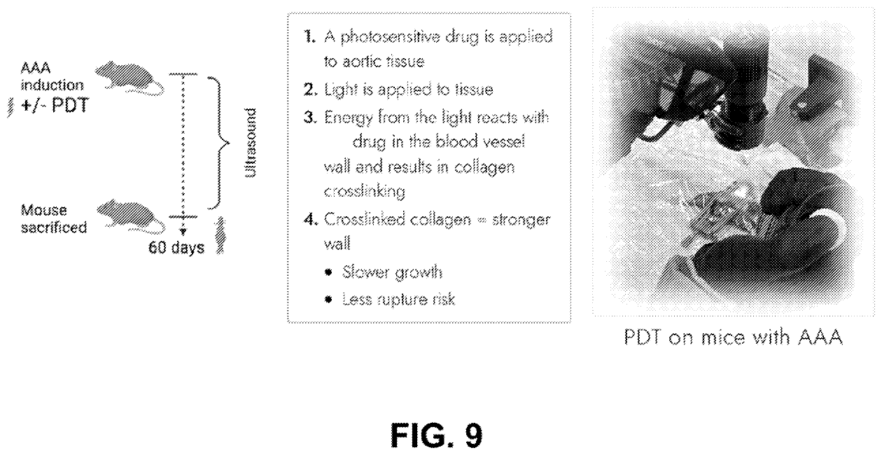

To assess if one of the improvements observed in aortic aneurysms upon MB PDT is due to an effect on the mechanical strength of the tissue, the regional healthy aortic tissue from the same region as MB PDT aneurysms occur in mice during experimental AAA surgery was subjected to PDT ex vivo. A forceps test was conducted to measure stiffness of the tissue. Interestingly, MB PDT-induced vascular stiffness increased with duration of light exposure. (FIG. 7A). The stiffness maximized at 120 mins exposure for mouse tissue. This benefit was consistent in porcine aorta treated with MB PDT (FIG. 7B), although the exposure requirement was higher and visually less striking. This maybe due to variability between elastin and collagen content within aortic tissue between different species. Additionally porcine aorta subjected to incubation with recombinant elastase was resistant to degradation after MB PDT treatment (FIG. 7B), with more resistance observed with higher intensity of light (60 mW versus 120 mW). To assess if this benefit of MB PDT on mechanical strength is true in a pathological setting, MB PDT treatment was done of on mouse aneurysms in vivo and the stiffness was assessed ex vivo (FIG. 7C). A clear improvement in the stiffness of the aneurysm was observed post-MB PDT treatment compared to vehicle treatment controls further suggesting that the observed benefit on mechanical properties of the tissue is clinically relevant.

As described above, the data collectively demonstrates that the photodynamic therapy using the activatable conjugate is able to achieve photoactivation of the photosensitizer that: (i) reduces or maintains the diameter of a blood vessel having an aneurysm, as compared to an untreated blood vessel having an aneurysm; (ii) decreases the volume of an aneurysm or inhibits an increase in the volume of the aneurysm, as compared to an untreated aneurysm; (iii) reduces the rate of blood vessel diameter growth in an blood vessel having an aneurysm, as compared to an untreated blood vessel having an aneurysm; (iv) decreases the rate of volume increase in an aneurysm or inhibits an increase in the volume of the aneurysm, as compared to an untreated aneurysm; and/or (v) is localized to regions of tissue degradation or tissue inflammation or within or near the site of an aneurysm (e.g., after parenteral administration), as compared to a region of healthy tissue, and/or increases crosslinking within the ECM.

### Methods—Tissue Rigidity Assay:

Treated tissues were tested for their rigidity and ability to resist drop due to gravity. First, a ruler (with a specificity up to mm) was taped vertically against a white background and checked with a water leveler. Next, each sample was carefully held with a forcep at 0 mm on the ruler, such that the sample would, if perfectly stiff, be perpendicular to the ruler. The forcep was also held as perpendicular to the ruler, such that the end of the tissue between the tips of the forcep was parallel to the forcep. Next the drop of the sample was quantified by measuring the distance the other end of the sample reached on the ruler. Each sample was also photographed and filmed for record and display purposes. The weight and length of each sample was noted. Mouse aneurysms and aortic tissue were treated with MB PDT and vehicle in vivo and ex vivo. Unless otherwise specified, the methods described in this section can be similarly applied to similar experiments throughout the examples.

While preferred embodiments of the present disclosure have been shown and described herein, it will be obvious to those skilled in the art that such embodiments are provided by way of example only. Numerous variations, changes, and substitutions will now occur to those skilled in the art without departing from the instant disclosure. It should be understood that various alternatives to the embodiments described herein may be employed in practicing the invention. It is intended that the following claims define the scope of the embodiments disclosed herein, and that methods and structures within the scope of these claims and their equivalents be covered thereby.

SEQUENCES

| Number | Sequence | Note |
|---|---|---|
| 1 | MGRPSGRKSSKMQAFRIWDVNQKTFYLRNNQLVAGYLQG | anakinra |
| | PNVNLEEKIDVVPIEPHALFLGIHGGKMCLSCVKSGDET | |
| | RLQLEAVNITDLSENRKQDKRFAFIRSDSGPTTSFESAA | |
| | CPGWFLCTAMEADQPVSLTNMPDEGVMVTKFYFQEDE | |
| 2 | APVRSLNCRIWDVNQKTFYLRNNQLVAGYLQGPNVNLEE | ebi005 |
| | KFSMSFVQGEESNDKIPVALGLKEKNLYLSCVLKDDKPT | |
| | LQLESVDPKNYPKKKMEKRFVENKIEINNKLEFESAQFP | |
| | NWFLCTAMEADQPVSLTNMPDEGVMVTKFYMQFVSS | |
| 3 | APVRSLNCRIWDVNQKTFYLRNNQLVAGYLQGPNVNLEE | Ebi005 |
| | KFSMSFVQGEESNDKIPVALGLKEKNLYLSCVLKDDKPT | |
| | LQLESVDPKNYPKKKMEKRFVENKIEINNKLEFESAQFP | |
| | NWFLCTAMEADQPVSLTNMPDEGVMVTK | |
| 4 | SERCDDWGLDTMRQIQVFEDEPARIKCPLFEHFLKENYS | Rilonacept |
| | TAHSAGLTLIWYWTRQDRDLEEPINFRLPENRISKEKDV | |
| | LWFRPTLLNDTGNYTCMLRNTTYCSKVAFPLEVVQKDSC | |
| | FNSPMKLPVHKLYIEYGIQRITCPNVDGYFPSSVKPTIT | |
| | WYMGCYKIQNENNVIPEGMNLSFLIALISNNGNYTCVVT | |
| | YPENGRTFHLTRTLTVKVVGSPKNAVPPVIHSPNDHVVY | |
| | EKEPGEELLIPCTVYFSFLMDSRNEVWWTIDGKKPDDIT | |
| | IDVTINESISHSRTEDETRTQILSIKKVTSEDLKRSYVC | |
| | HARSAKGEVAKAAKVKQKVPAPRYTVEKCKEREEKIILV | |
| | SSANEIDVRPCPLNPNEHKGTITWYKDDSKTPVSTEQAS | |
| | RIHQHKEKLWFVPAKVEDSGHYYCVVRNSSYCLRIKISA | |
| | KFVENEPNLCYNAQAIFKQKLPVAGDGGLVCPYMEFFKN | |
| | ENNELPKLQWYKDCKPLLLDNIHFSGVKDRLIVMNVAEK | |
| | HRGNYTCHASYTYLGKQYPITRVIEFITLEENKPTRPVI | |
| | VSPANETMEVDLGSQIQLICNVTGQLSDIAYWKWNGSVI | |
| | DEDDPVLGEDYYSVENPANKRRSTLITVLNISEIESRFY | |
| | KHPFTCFAKNTHGIDAAYIQLIYPVTNSGDKTHTCPPCP | |
| | APELLGGPSVFLFPPKPKDTLMISRTPEVTCVVVDVSHE | |
| | DPEVKENWYVDGVEVHNAKTKPREEQYNSTYRVVSVLTV | |
| | LHQDWLNGKEYKCKVSNKALPAPIEKTISKAKGQPREPQ | |
| | VYTLPPSRDELTKNQVSLTCLVKGFYPSDIAVEWESNGQ | |
| | PENNYKTTPPVLDSDGSFFLYSKLTVDKSRWQQGNVFSC | |
| | SVMHEALHNHYTQKSLSLSPGK | |
| 5 | QVQLVESGGGVVQPGRSLRLSCAASGFTFSVYGMNWVRQ | Canakinumab |
| | APGKGLEWVAIIWYDGDNQYYADSVKGRFTISRDNSKNT | _vh |
| | LYLQMNGLRAEDTAVYYCARDLRTGPFDYWGQGTLVTVS | |
| | S | |
| 6 | EIVLTQSPDFQSVTPKEKVTITCRASQSIGSSLHWYQQK | Canakinumab |
| | PDQSPKLLIKYASQSFSGVPSRFSGSGSGTDFTLTINSL | _vl |
| | EAEDAAAYYCHQSSSLPFTFGPGTKVDIKRTV | |
| 7 | QVQLQESGPGLVKPSQTLSLTCSFSGESLSTSGMGVGWI | Gevokizumab |
| | RQPSGKGLEWLAHIWWDGDESYNPSLKSRLTISKDTSKN | _vh |
| | QVSLKITSVTAADTAVYFCARNRYDPPWFVDWGQGTLVT | |
| | VSS | |
| 8 | DIQMTQSTSSLSASVGDRVTITCRASQDISNYLSWYQQK | Gevokizumab |
| | PGKAVKLLIYYTSKLHSGVPSRESGSGSGTDYTLTISSL | _v1 |
| | QQEDFATYFCLQGKMLPWTFGQGTKLEIKRTV | |
| 9 | VQLVQSGAEVKKPGSSVKVSCKASGYAFTSSWMNWVRQA | Nadunolimab |
| | PGQGLEWMGRIYPGDGNTHYAQKFQGRVTLTADKSTSTA | _vh |
| | YMELSSLRSEDTAVYYCGEGYLDPMDYWGQGTLVT | |
| 10 | DIQMTQSPSSLSASVGDRVTITCQASQGINNYLNWYQQK | Nadunolimab |
| | PGKAPKLLIHYTSGLHAGVPSRESGSGSGTDYTLTISSL | _v1 |
| | EPEDVATYYCQQYSILPWTFGGGTKVEIK | |

1. A photoactivatable drug conjugate comprising anakinra attached to a photosensitizer is activated when exposed to light at a wavelength of about 600 nm to about 1000 nm.

2. The photoactivatable drug conjugate of claim 1, wherein photoactivation of the photosensitizer:
(i) reduces or maintains the diameter of a blood vessel having an aneurysm, as compared to an untreated blood vessel having an aneurysm;
(ii) decreases the volume of an aneurysm or inhibits an increase in the volume of the aneurysm, as compared to an untreated aneurysm;
(iii) reduces the rate of blood vessel diameter growth in an blood vessel having an aneurysm, as compared to an untreated blood vessel having an aneurysm; and/or
(iv) decreases the rate of volume increase in an aneurysm or inhibits an increase in the volume of the aneurysm, as compared to an untreated aneurysm.

3. The photoactivatable drug conjugate of any one of claims 1-2, wherein after parenteral administration, the photoactivatable drug conjugate localizes to regions near or within tissue inflammation or an aneurysm, as compared to a region of healthy tissue.

4. The photoactivatable drug conjugate of any one of claims 1-3, wherein the photosensitizer is selected from: porfimer sodium, a porphyrin, a chlorin, a pheophorbide, a bacteriopheophorbide, a phthalocyanine, a anthraquinone, a phenothiazine, a xanthene, or a cyanine; optionally wherein the photosensitizer is selected from Table 1.

5. The photoactivatable drug conjugate of any one of claims 1-4, wherein the photosensitizer generates singlet oxygen when exposed to light at a wavelength of about 600 nm to about 1000 nm.

6. A photoactivatable drug conjugate comprising anakinra attached via a linker to methylene blue.

7. The photoactivatable drug conjugate of any one of claims 1-6, wherein the anakinra comprises an amino acid sequence having at least 85%, 90%, 95%, 97%, 98%, 99%, or greater sequence identity to SEQ ID NO: 1, wherein the anakinra binds IL1R.

8. The photoactivatable drug conjugate of claim 7, wherein the anakinra comprises an amino acid sequence of SEQ ID NO: 1.

9. The photoactivatable drug conjugate of any one of claims 1-8, wherein the photosensitizer is covalently attached to the anakinra.

10. The photoactivatable drug conjugate of any one of claims 1-9, wherein the photosensitizer is attached to the targeting moiety via a linkage comprising an amide linkage, an oxime linkage, triazole linkage, or a thioether linkage.

11. The photoactivatable drug conjugate of any one of claims 1-10, wherein the photosensitizer is attached to a cysteine residue or a lysine residue of the anakinra.

12. The photoactivatable drug conjugate of any one of claims 1-11, wherein the photosensitizer is attached to one or more cysteine residues or one or more lysine residues of the anakinra.

13. The photoactivatable drug conjugate of any one of claims 1-12, wherein the photoactivatable drug conjugate comprises a plurality of photosensitizers attached to the anakinra.

14. A photoactivatable drug conjugate comprising a photosensitizer attached to a targeting moiety, wherein the targeting moiety binds an inflamed tissue.

15. The photoactivatable drug conjugate of claim 14, wherein the inflamed tissue is a vascular tissue.

16. The photoactivatable drug conjugate any one of claims 14-15, wherein the inflamed tissue is a blood vessel.

17. The photoactivatable drug conjugate any one of claims 14-16, wherein the inflamed tissue is within or near the site of an aneurysm.

18. The photoactivatable drug conjugate any one of claims 14-17, wherein the targeting moiety binds to a cytokine receptor or an inflammatory molecule.

19. The photoactivatable drug conjugate of any one of claims 14-18, wherein photoactivation of the photosensitizer:
(i) reduces or maintains the diameter of a blood vessel having an aneurysm, as compared to an untreated blood vessel having an aneurysm;
(ii) decreases the volume of an aneurysm or inhibits an increase in the volume of the aneurysm, as compared to an untreated aneurysm;
(iii) reduces the rate of blood vessel diameter growth in an blood vessel having an aneurysm, as compared to an untreated blood vessel having an aneurysm; and/or
(iv) decreases the rate of volume increase in an aneurysm or inhibits an increase in the volume of the aneurysm, as compared to an untreated aneurysm.

20. The photoactivatable drug conjugate of any one of claims 14-19, wherein the photoactivatable drug conjugate localizes to regions near or within tissue inflammation or an aneurysm, as compared to a region of healthy tissue.

21. The photoactivatable drug conjugate of any one of claims 14-20, wherein the photosensitizer generates singlet oxygen when exposed to light at a wavelength of about 350 nm to about 1200 nm.

22. The photoactivatable drug conjugate of any one of claims 14-21, wherein the photosensitizer generates singlet oxygen when exposed to light at a wavelength of about 600 nm to about 1000 nm.

23. The photoactivatable drug conjugate of any one of claims 14-22, wherein the photosensitizer is selected from: porfimer sodium, a porphyrin, a chlorin, a pheophorbide, a bacteriopheophorbide, a phthalocyanine, a anthraquinone, a phenothiazine, a xanthene, or a cyanine; optionally wherein the photosensitizer is selected from Table 1.

24. The photoactivatable drug conjugate of any one of claims 14-23, wherein the photosensitizer is methylene blue.

25. The photoactivatable drug conjugate of any one of claims 14-23, wherein the photosensitizer is indocyanine green.

26. The photoactivatable drug conjugate of any one of claims 14-25, wherein the photosensitizer is covalently attached to the targeting moiety.

27. The photoactivatable drug conjugate of any one of claims 14-26, wherein the photosensitizer is attached to the targeting moiety via a linkage comprising an amide linkage, an oxime linkage, triazole linkage, or a thioether linkage.

28. The photoactivatable drug conjugate of any one of claims 14-27, wherein the photosensitizer is attached to a cysteine residue or a lysine residue of the targeting moiety.

29. The photoactivatable drug conjugate of any one of claims 14-28, wherein the photosensitizer is attached to one or more cysteine residues or one or more lysine residues of the targeting moiety.

30. The photoactivatable drug conjugate of any one of claims 14-29, wherein the photoactivatable drug conjugate comprises a plurality of photosensitizers attached to the targeting moiety.

31. The photoactivatable drug conjugate of any one of claims 14-30, wherein the targeting moiety is selected from: an antibody or antibody fragment, a protein, a ligand, or a small molecule.

32. The photoactivatable drug conjugate of any one of claims 14-31, wherein the targeting moiety binds IL1R.

33. The photoactivatable drug conjugate of any one of claims 14-32, wherein the targeting moiety comprises an amino acid sequence having at least 90% sequence identity to any one of SEQ ID NOS: 1-4, wherein the targeting moiety binds IL1R.

34. The photoactivatable drug conjugate of any one of claims 14-33, wherein the targeting moiety comprises an amino acid sequence of any one of SEQ ID NOs: 1-4.

35. The photoactivatable drug conjugate of any one of claims 14-34, wherein the targeting moiety binds IL1beta.

36. The photoactivatable drug conjugate of claim 35, wherein the targeting moiety is an antibody comprising a heavy chain variable domain having at least 90% sequence identity to SEQ ID NO: 5 and a light chain variable domain having at least 90% sequence identity to SEQ ID NO: 6.

37. The photoactivatable drug conjugate of claim 35, wherein the targeting moiety is an antibody comprising a heavy chain variable domain having at least 90% sequence identity to SEQ ID NO: 7 and a light chain variable domain having at least 90% sequence identity to SEQ ID NO: 8.

38. A pharmaceutical composition comprising the photoactivatable drug conjugate of any one of claims 1-37 and a pharmaceutically acceptable excipient.

39. A photodynamic therapy system comprising:
the photoactivatable drug conjugate of any one of claims 1-37;
a light delivery unit;
and a light generating unit comprising a light source.

40. The system of claim 39, wherein the light delivery unit comprises an optical applicator.

41. The system of claim 40, wherein the optical applicator comprises a catheter configured to deliver light.

42. The system of claim 41, wherein the catheter contains optical fibers to deliver the light.

43. The system of any one of claims 39-42, wherein the light generating unit generates light having a wavelength of about 600 nm to about 1000 nm.

44. The system of any one of claims 39-43, wherein the light delivery unit is capable of delivering light to tissue of an individual.

45. The system of claim 44, wherein the tissue is within or near the site of an aneurysm.

46. The system of claim 44, wherein the light delivery unit is capable of delivering light to a blood vessel.

47. The system of claim 44, wherein the light delivery unit is capable of delivering light to the aorta.

48. A kit comprising:
the photoactivatable drug conjugate of any one of claims 1-37; and
a light delivery unit of any one of claims 39-47.

49. A method of treating an aneurysm in an individual, the method comprising:
(a) administering a photosensitizer; and
(b) photoactivating the photosensitizer at the site of or near the aneurysm.

50. The method of claim 49, wherein the photosensitizer comprises the photosensitizer of any one of claims 1-37.

51. A method of treating an aneurysm in an individual, the method comprising:
(a) administering the photoactivatable drug conjugate of any one of claims 1-37 to the individual; and
(b) photoactivating the photosensitizer of the photoactivatable drug conjugate.

52. The method of claim 51, wherein said photoactivating occurs at the site of or near the aneurysm.

53. The method of any one of claims 51-52, wherein the photoactivatable drug conjugate localizes to regions near or within tissue inflammation or an aneurysm, as compared to a region of healthy tissue.

54. The method of any one of claims 49-53, wherein photoactivating the photosensitizer:
(i) reduces or maintains the diameter of a blood vessel having an aneurysm, as compared to an untreated blood vessel having an aneurysm;
(ii) decreases the volume of an aneurysm or inhibits an increase in the volume of the aneurysm, as compared to an untreated aneurysm;
(iii) reduces the rate of blood vessel diameter growth in an blood vessel having an aneurysm, as compared to an untreated blood vessel having an aneurysm; and/or
(iv) decreases the rate of volume increase in an aneurysm or inhibits an increase in the volume of the aneurysm, as compared to an untreated aneurysm.

55. The method of claim 54, wherein the blood vessel is the aorta.

56. The method of any one of claims 49-55, wherein the aneurysm is an aortic aneurysm, a cerebral aneurysm, a thoracic aortic aneurysm, an abdominal aortic aneurysm, a popliteal artery aneurysm, a peripheral aneurysm, a fusiform aneurysm, a saccular aneurysm, a mycotic aneurysm, a pseudo aneurysm.

57. The method of any one of claims 49-56, wherein the aneurysm is an aortic aneurysm.

58. The method of any one of claims 49-57, wherein photoactivating in (b) is achieved by a catheter.

59. The method of claim 58, wherein the catheter is inserted through the groin artery.

60. The method of any one of claims 49-59, wherein the photosensitizer or photoactivatable drug conjugate is administered by parenteral injection.

61. The method of claim 60, wherein the photosensitizer or photoactivatable drug conjugate is administered by intravenous injection.

62. A method for treating an aneurysm comprising: administering to a subject in need thereof an activatable drug conjugate wherein the activatable drug conjugate comprises a targeting moiety for targeting the conjugate to an inflamed tissue and an activatable agent that is activated via an energy source; and administering energy from the energy source to a site at or near the aneurysm.
